(19) United States
(12) Patent Application Publication
(10) Pub. No.: US 2026/0220495 A1
(43) Pub. Date: Jul. 30, 2026

(54) Data Structures for Use in Clinical Decision Making Systems

(71) Applicant: NEOM Company, Riyadh (SA)

(72) Inventor: Hany Elsayed OSMAN, Riyadh (SA)

(21) Appl. No.: 19/574,099

(22) Filed: Mar. 20, 2026

Related U.S. Application Data
(63) Continuation of application No. PCT/IB2025/058688, filed on Aug. 28, 2025.

(30) Foreign Application Priority Data
Aug. 30, 2024 (GB) GB 2412792.0

(51) Int. Cl. G06N 5/022; G16H 10/60; G16H 50/20

(52) U.S. Cl. CPC G06N 5/022; G16H 10/60; G16H 50/20

(57) ABSTRACT

A computer implemented method for converting medical data into a graph structure for improved diagnostic querying comprises: converting first information on a first health condition into a first sub-knowledge-graph, sub-KG, the first sub-KG having the following structure: a first layer comprising a first root node that is activated based on a first health indicator related to the first health condition; a second layer of nodes wherein each node in the second layer of nodes indicates a criteria to be applied to the first health indicator indicated in the root node; and a third layer of nodes, each node in the third layer of nodes being linked to a respective node in the second layer of nodes and corresponding to an interpretation associated with the first health indicator satisfying the criteria of the respective second layer node to which it is linked. The method further comprising: integrating the first sub-KG into a global knowledge-graph GKG, the GKG having a global root node and a first layer of nodes linked to the global root node; and wherein the step of integrating the first sub-KG into the GKG comprises adding the root node of the first sub-KG to the first layer of the GKG.

### RELATED APPLICATIONS

This application is a bypass continuation of International Application No. PCT/IB2025/058688, filed Aug. 28, 2025, entitled “Data Structures for Use in Clinical Decision Making Systems” which claims priority to United Kingdom Patent Application No. 2412792.0, filed Aug. 30, 2024, entitled “Data Structures for Use in Clinical Decision Making Systems” which are incorporated by reference herein in their entirety.

### FIELD OF PRESENT DISCLOSURE

The present disclosure relates to systems and methods for converting medical data into a new graph structure that allows for improved diagnostic querying.

### BACKGROUND

Most healthcare systems are designed for Western patient population cohorts and have biases with respect to factors such as ethnicity and gender. Furthermore, they consider a narrow range of inputs, often missing factors such as the environment that the person is exposed to, e.g. pollutants and sun-exposure, and factors relating to their genetics.

Many existing healthcare systems include predictive models, wherein the predictive models vary from a simple sequence of ‘if-then’ statements to more complex models such as machine learning-based models.

At the heart of such systems lies the Electronic Healthcare Record (EHR) that is used to record patient data, and the Electronic Medical Record (EMR) which is used at a practice management level.

These systems are known to have wide-ranging limitations, including incomplete patient history and diagnostic information; inaccurate or incomplete diagnoses; variable clinical care; patient risk from disjointed care coordination; poor patient engagement; and a limited understanding of disease prevalence and chronic conditions within regional and local populations. A number of these limitations arise as a result of scattered records, an inability to consolidate patient information, and a lack of interoperability across systems.

As a result, many healthcare professionals (surgeons, doctors, nurses, and any other clinicians) see EHRs as merely information storage and do not consider EHRs to result in better healthcare outcomes for their patients.

### SUMMARY OF THE DISCLOSURE

As noted above, patient clinical data is typically stored in an EHR or EMR. Such data records often contain a chronological set of notes made by the healthcare professions that they have interacted with, describing the outcomes of appointments and the like, as well as the results of any tests performed on the patient. The data is stored in a patient-oriented or patient-centric manner.

Healthcare professionals use the information in the EHR, together with their knowledge and experience, to diagnose the patient's medical conditions. However, a single clinician's ability to diagnose a patient is limited by their knowledge and experience, or their access to relevant information. Furthermore, EHRs are sub-optimally designed and it can be laborious to scroll through reams of text-based notes from previous examinations to find relevant information.

More recently, Machine Learning (ML) has been used to predict diagnoses and to make recommendations from EHRs. These systems often work in a “black-box” manner however, which makes it difficult for healthcare professionals to interpret how the ML model came to a particular result. This black-box nature leads to loss of confidence in such systems and thus poor usage uptake.

More significantly, most ML solutions, such as neural network-based solutions are non-deterministic, as the predictions made are not necessarily repeatable, even when the same underlying data is passed to the model under the same conditions. The lack of repeatability/stability and explainabilty is a significant limitation of these types of systems.

Another issue is the disparate sources of medical available to a healthcare professional. Collating information from medical text books, academic research papers and disparate online resources is an expensive and impractical task.

Knowledge graphs (KGs) have been explored for aggregating and representing large amounts of medical data e.g. information on thousands of diseases, into a single access point. KGs have not tended to be easily scalable however, requiring prohibitively large amounts of manual work and input by healthcare professionals to clinically validate information in order to integrate new clinical scenarios into a single KG.

Whilst KGs can comprise vast amounts of data in a structured format, the interconnectivity of diseases through common medical indicators e.g. genetic indicators or clinical test results, leads to highly complex graph structures that make manual interaction with the graph an arduous task.

Additionally, the sector of medicine is continuously evolving. For example, new data related to the understanding of diseases, methods of treatment, and drug production appear on a near-daily basis. Thus, given the manual and laborious nature adding new information to a KG, keeping a KG up to date in an efficient and accurate manner poses a significant challenge.

Another challenge in creating clinically validated KGs is the selection of appropriate node types and vocabulary used in the KG. KGs of medicine are usually generated using many different and diverse data sources and the vocabulary varies between the different data sources. The vocabulary thus has to be matched and standardised manually by clinicians. As a result, matching clinical insights and diagnostic tools in diverse data sources to ensure that all available information has been captured in the KG duplicates is a time-consuming process and can lead to imperfections.

Embodiments described herein address at least some of these challenges through the use of a new structure of KG and through new, automatable methods that facilitate the production of a highly reliable, yet scalable, KG.

In a first aspect herein, there is a computer implemented method for converting medical data into a graph structure for improved diagnostic querying. The method comprises: converting first information on a first health condition into a first sub-knowledge-graph, sub-KG. The first sub-KG has the following structure: a first layer comprising a first root node that is activated based on a first health indicator related to the first health condition; a second layer of nodes wherein each node in the second layer of nodes indicates a criteria to be applied to the first health indicator indicated in the root node; and a third layer of nodes, each node in the third layer of nodes being linked to a respective node in the second layer of nodes and corresponding to an interpretation associated with the first health indicator satisfying the criteria of the respective second layer node to which it is linked. The method then comprises integrating the first sub-KG into a global knowledge-graph (GKG). The GKG has a global root node and a first layer of nodes linked to the global root node, and the step of integrating the first sub-KG into the GKG comprises adding the root node of the first sub-KG to the first layer of the GKG.

The aforementioned structure has a range of advantages. The resulting KG is disease-centric, to the extent that it essentially represents a disease-digital twin, capturing all information on diseases in a single, stratified format.

The structure herein is scalable, as each sub-graph is structured such that it can be integrated into the global KG in an efficient way, because the design of the structure means that the root of each sub-graph forms a node in the first layer of the global-KG (or GKG) and can be effectively slotted in. Thus, the global-KGs herein can be used to unify information from diverse data sources into a single, standardised format. The global KGs described herein can potentially thus be used as a single unified repository of all medical knowledge.

Furthermore, the global-KG is easily updatable as new clinical information is available. Because the global-KG is stratified into sub-KGs, each sub-KG dealing with an individual health indicator, when an update to said health indicator is found, then the respective sub-KG can be updated and re-integrated/slotted back in to the larger global-KG. This has the advantage that the entire global-KG does not need to be re-evaluated each time a clinical update is obtained. Thus, the structure herein allows for regular updating in light of new clinical information, in a reliable and structured manner.

Furthermore, when in use, the structure herein permits more efficient queries of the data held within it. Each sub-KG is separate from each other sub-graph, and thus, each sub-graph can be queried (e.g. compared to patient data) in parallel, leading to fast, efficient queries, whereby patient data is methodically and robustly compared to a multitude of different disease profiles in a thorough, repeatable and reliable manner.

In some embodiments, the method further comprises converting second information on a second health condition into a second sub-KG, the second sub-KG having the same structure as the first sub-KG, and integrating the second sub-KG into the global-KG, wherein the step of integrating the second sub-KG into the global-KG comprises adding the root node of the second sub-KG to the first layer of the global-KG.

In some embodiments, the method further comprises the step of converting the first information on the first disease into the first sub-KG comprises prompting a large language model, LLM, to convert the first information into the first sub-KG, wherein the prompt used provides the first information and a description of the structure that the first sub-KG should take to the LLM.

In some embodiments, the method further comprises comparing the output of the LLM to a gold-standard sub-KG.

In some embodiments, directional stimulus prompting, DSP, is used to form the prompt.

In an embodiment, retrieval augmented generation, RAG, is further used in combination with the DSP to form the prompt.

In some embodiments, the first layer, the second layer and the third layer of the first sub-KG and the GKG are unidirectional, with no reverse edges.

In embodiments, the first sub-KG further comprises one or more further layers, the nodes in the one or more further layers representing different explanations for each interpretation of a respective third layer node.

In some embodiments, the first health indicator is a laboratory test that can be performed on a patient, the state of the first health indicator indicates a first range of values of the first health indicator, and the interpretation of the state of the first health indicator is a medical condition that can be inferred from a laboratory test result with the first range of values of the first health indicator.

In some embodiments, the first health indicator relates to a genetic test that can be performed on a patient a socio-economic health indicator, or an environmental health indicator.

In some embodiments, the first health indicator relates to a datum collected during a clinical visit, or a datum collected using a remote patient monitoring device.

In some embodiments, the method further comprises receiving an update to the first information, converting the updated first information into an updated first sub-KG, the updated first sub-KG having the same structure as the first sub-KG, and replacing the updated first sub-KG into the global-KG in place of the first sub-KG.

In some embodiments, the method further comprises outputting the GKG, and storing the GKG in a database.

In a second aspect, there is provided an apparatus for converting medical data into a graph structure for improved diagnostic querying, comprising a memory comprising instruction data representing a set of instructions, and a processor configured to communicate with the memory and to execute the set of instructions, wherein the set of instructions, when executed by the processor, cause the processor to convert first information on a first health condition into a first sub-knowledge-graph, sub-KG, and to integrate the first sub-KG into a global knowledge-graph GKG, the GKG having a global root node and a first layer of nodes linked to the global root node, and wherein the processor is caused to integrate the first sub-KG into the GKG by adding the root node of the first sub-KG to the first layer of the GKG. The first sub-KG has the structure with a first layer comprising a first root node that is activated based on a first health indicator related to the first health condition, a second layer of nodes wherein each node in the second layer of nodes indicates one or more criteria to be applied to the first health indicator indicated in the root node, and a third layer of nodes, each node in the third layer of nodes being linked to a respective node in the second layer of nodes and corresponding to an interpretation associated with the first health indicator satisfying the criteria of the respective second layer node to which it is linked.

In some embodiments, the processor is further caused to perform a computer implemented method for converting medical data into a graph structure for improved diagnostic querying in various aspects and embodiments.

In a third aspect, there is provided a data structure for storing medical data for improved diagnostic querying, the data structure comprising a global-knowledge-graph, global-KG, comprising, a plurality of sub-knowledge graphs, sub-KGs. Each sub-KG has the structure with a first layer comprising a first root node that is activated based on a first health indicator related to the first health condition, a second layer of nodes wherein each node in the second layer of nodes indicates one or more criteria to be applied to the first health indicator indicated in the root node, and a third layer of nodes, each node in the third layer of nodes being linked to a respective node in the second layer of nodes and corresponding to an interpretation associated with the first health indicator satisfying the criteria of the respective second layer node to which it is linked, wherein the global-KG has a global root node and a first layer of nodes linked to the global root node, and wherein the plurality of sub-graphs are integrated into the global-KG by means of their respective root nodes having been added to the first layer of the global-KG.

In a fourth aspect, the present disclosure provides a computer program comprising instructions which, when executed by a computer cause the computer to perform the computer implemented method of the first aspect.

In the fifth aspect, the present disclosure provides a computer readable storage medium comprising instructions which, when executed by a computer, cause the computer to carry out the method of the first aspect.

According to a sixth aspect herein, there is provided a method of use of the global-KG with the aforementioned structure. A method of processing patient data to obtain a clinical insight, comprises: i) parsing the patient data through a global-knowledge graph, global-KG. The global-KG comprises: a plurality of sub-knowledge graphs, sub-KGs, each sub-KG having the following structure: a first layer comprising a first root node that is activated based on a first health indicator related to a first health condition; a second layer of nodes wherein each node in the second layer of nodes indicates a criteria to be applied to the first health indicator indicated in the root node; and a third layer of nodes, each node in the third layer of nodes being linked to a respective node in the second layer of nodes and corresponding to an interpretation associated with the first health indicator satisfying the criteria of the respective second layer node to which it is linked. The global-KG has a global root node and a first layer of nodes linked to the global root node, and wherein the plurality of sub-KGs are integrated into the global-KG by means of their respective root nodes having been added to the first layer of the global-KG. The method further comprises: obtaining the clinical insight from the nodes in the global-KG that are activated when the converted patient data is parsed through the global-KG.

The global-KG is capable of acting as a true Knowledge Graph of Medicine or disease digital twin. The aforementioned scalability of the structure described herein allows for potentially unlimited medical conditions to be encompassed therein in a single stratified manner. When in use, this has the advantage of processing the patient data in a reliable and robust way to determine whether the patient has any of the underlying conditions or risk factors encompassed therein.

This has the potential to move clinical diagnostics from a patient-centric model where limited (and potentially incomplete) information about the patient is the focus of the diagnostic process, to a disease-oriented diagnostic process.

Use of the global-KG as described herein is deterministic, in that the results are repeatable and produce the same results when the same data is input to the system.

The output of the global-KG is fully explainable, due to the structure of the global-KG which provides indications and explanations as part of its output. These two features (that the output is deterministic and explainable) enable a clinician to model different scenarios to determine how this changes the resulting clinical insights or diagnoses. Thus, leading to improved clinical understanding and ultimately to improved patient outcomes.

In some embodiments, the patient data is parsed through each sub-KG in parallel.

In some embodiments, the patient data is parsed through the global-KG using a depth-first-search, DFS, method.

In some embodiments, the method further comprises setting an activation parameter, so as to mark whether each respective node has been activated when the patient data is parsed through the global-knowledge graph, and saving a sub-set of the nodes in the global-KG that were activated, as a patient-specific KG.

In some embodiments, the method further comprises removing one or more nodes in the global-KG that were not activated when the patient data is parsed through the global-knowledge graph.

In some embodiments, the method further comprises providing an instruction to a display to display the patient-specific KG, the instruction further comprising instructions to annotate each node and edge in the patient-specific KG.

In some embodiments, the instruction further instructs the display to mark a first subset of nodes in the first layer of nodes as Findings, a second subset of nodes in the second layer of nodes as Interpretations, and a third subset of nodes in the third layer of nodes as Explanations.

In some embodiments, the method further comprises receiving updated patient data, and repeating the steps of parsing the patient data through a global-knowledge graph, and obtaining the clinical insight from the nodes in the global-KG that are activated when the converted patient data is parsed through the global-KG, on the updated patient data.

In some embodiments, the method further comprises, preceding steps i) and ii), converting the patient data into a structured data format.

In some embodiments, the method further comprises receiving, at one or more nodes in the third layer of nodes, prevalence statistics relating to the interpretation associated with the first health indicator.

In the seventh aspect, the present disclosure provides an apparatus for processing patient data to obtain a clinical insight, comprising a memory comprising instruction data representing a set of instructions, and a processor configured to communicate with the memory and to execute the set of instructions, wherein the set of instructions, when executed by the processor, cause the processor to perform the of the sixth aspect.

In the eighth aspect, the disclosure provides a computer program comprising instructions which, when executed by a computer cause the computer to perform the method of the sixth aspect; hold in memory a patient specific knowledge graph; or display a patient specific knowledge graph.

In the ninth aspect, the present disclosure provides a computer readable storage medium comprising instructions which when executed by a computer cause the computer to carry out the method of the sixth aspect.

In the tenth aspect, the present disclosure provides a digital healthcare system comprising a clinician-facing application, a patient-facing application, and a server-based application, wherein the patient-facing application is configured to receive in a first user input, first patient data; and send the first patient data to the server-based application. The server-based application is configured, upon receiving the first patient data, to trigger the first patient data to be parsed through a global knowledge-graph (KG) comprising a plurality of sub-KGs, each sub-KG relating to a different health condition, receive a first clinical insight as output from the global-KG; and send the first clinical insight to the clinician-facing application, and wherein the clinician-based application is configured to display a first update, in response to receiving the first clinical insight.

In some embodiments, the clinician-facing application is configured to receive in a second user input, second patient data, and send the second patient data to the server-based application, wherein the server-based application is configured, upon receiving the second patient data, to trigger the second patient data to be parsed through the global-KG, receive a second clinical insight as output from the global-KG; and send the second clinical insight to the patient-facing application; and wherein the patient-based application is configured to display a second update for the patient, in response to receiving the second clinical insight.

In some embodiments, the clinician-facing application is further configured to receive in a third user input, third patient data, and send the third patient data to the server-based application, receive a third clinical insight as output from the global-KG, and display a comparison of the third clinical insight, with one or more previous clinical insights obtained for the patient.

In some embodiments, the third clinical insight is displayed as a patient-specific KG, the patient-specific KG comprising a subset of nodes from the global-KG that are activated when the third patient data is parsed through the global-KG.

In some embodiments, the comparison is displayed as a change to the patient-specific KG.

In some embodiments, the plurality of sub-KGs has the structure with a first layer comprising a first root node that is activated based on a first health indicator related to a first health condition, a second layer of nodes wherein each node in the second layer of nodes indicates a criteria to be applied to the first health indicator indicated in the root node, and a third layer of nodes, each node in the third layer of nodes being linked to a respective node in the second layer of nodes and corresponding to an interpretation associated with the first health indicator satisfying the criteria of the respective second layer node to which it is linked. The global-KG has a global root node and a first layer of nodes linked to the global root node, and wherein the plurality of sub-KGs are integrated into the global-KG by means of their respective root nodes having been added to the first layer of the global-KG.

In the eleventh aspect, the present disclosure provides a clinician-facing apparatus comprising a memory comprising instruction data representing a set of instructions, and a processor configured to communicate with the memory and to execute the set of instructions, wherein the set of instructions, when executed by the processor, cause the processor to receive a first clinical insight, wherein the first clinical insight is received from a server-based application, in response to the server-based application receiving first patient data from a user input provided by the patient, and the server-based application being triggered to parse the first patient data through a global knowledge-graph (KG), comprising a plurality of sub-KGs, each sub-KG relating to a different health condition, to receive the first clinical insight.

In some embodiments, the clinician-facing apparatus is further configured to receive in a second user input, second patient data, and send the second patient data to the server-based application, and in response, receive from the server-based application, a second clinical insight, the second clinical insight having been obtained by parsing the second patient data through the global-KG.

In some embodiments, the clinician-facing apparatus is further configured to receive in a third user input, third patient data, and send the third patient data to the server-based application, receive a third clinical insight as output from the global-KG; and display a comparison of the third clinical insight, with one or more previous clinical insights obtained for the patient.

In some embodiments, the third clinical insight is displayed as a patient-specific KG, the patient-specific KG comprising a subset of nodes from the global-KG that were activated when the third patient data was parsed through the global-KG.

In some embodiments, the comparison is displayed as a change to the patient-specific KG.

In the twelfth aspect, the present disclosure provides a patient-facing apparatus comprising a memory comprising instruction data representing a set of instructions, and a processor configured to communicate with the memory and to execute the set of instructions, wherein the set of instructions, when executed by the processor, cause the processor to receive in a first user input, first patient data, and send the first patient data to a server based application to trigger the server-based application to parse the first patient data through a global knowledge-graph (KG), comprising a plurality of sub-KGs, each sub-KG relating to a different health condition, and receive, in response, a first clinical insight from the server-based application, from the output of the global-KG.

In some embodiments, the processor is further configured to receive a second clinical insight, wherein the second clinical insight is received from the server-based application, in response to the server-based application receiving first patient data from a user input provided by a clinician, and the server-based application being triggered to parse the first patient data through a global knowledge-graph, KG, comprising a plurality of sub-KGs, each sub-KG relating to a different health condition, to receive the second clinical insight.

In the thirteenth aspect, the present disclosure provides a method in a digital healthcare system, the method comprising: a patient-facing application receiving in a first user input, first patient data, and sending the first patient data to a server-based application. The server-based application, upon receiving the first patient data, triggering the first patient data to be parsed through a global knowledge-graph, KG, comprising a plurality of sub-KGs, each sub-KG relating to a different health condition. The server-based application, further receiving a first clinical insight as output from the global-KG, and sending the first clinical insight to a clinician-facing application, and the clinician-based application, in response, displaying a first update in response to receiving the first clinical insight.

In the fourteenth aspect, the present disclosure provides a computer program comprising instructions which, when executed by a computer cause the computer to perform the method of the thirteenth aspect.

In the fifteenth aspect, the present disclosure provides a computer readable storage medium comprising instructions which when executed by a computer cause the computer to carry out the method of the thirteenth aspect.

### BRIEF DESCRIPTION OF THE DRAWINGS

For a better understanding and to show more clearly how embodiments herein may be carried into effect, reference will now be made, by way of example only, to the accompanying drawings, in which:

FIG. 1 is a block diagram showing an example computer node according to some embodiments herein;

FIG. 2 is a flow diagram showing an example method for converting medical data into a graph structure for improved diagnostic querying;

FIG. 3 is a node diagram showing an example sub-KG having a structure according to embodiments herein;

FIG. 4 is a node diagram showing the structure of an example global-KG according to some embodiments herein;

FIG. 5 is a node diagram showing an example sub-KG output by an LLM according to some embodiments;

FIG. 6 is a node diagram showing an example sub-KG output by an LLM according to some embodiments;

FIG. 7 is a schematic block diagram showing a system for creating the global-KGs described herein;

FIG. 8 is a flow diagram showing a method according to some embodiments herein;

FIG. 9 is a flow diagram showing a method according to some embodiments herein;

FIG. 10 is a schematic block diagram showing a system for using the global-KGs described herein;

FIG. 11 is a node diagram showing an example of an alternative structure of the global-KG shown in FIG. 4;

FIG. 12 is a schematic flow diagram showing an initial state of a patient-specific KG according to some embodiments herein;

FIG. 13 is a schematic flow diagram showing a further state of a patient-specific KG after adding an encounter layer and modifying inter-relationships, as another embodiment of the patient-specific KG shown in FIG. 12;

FIG. 14 is a schematic block and node diagram showing an example of another alternative structure of the global-KG shown in FIG. 4;

FIG. 15 is a node diagram showing an example of another alternative structure of the global-KG shown in FIG. 4;

FIG. 16 is a schematic block diagram showing a digital healthcare system according to some embodiments herein;

FIG. 17 is a flow diagram showing a method of operating the digital healthcare system of FIG. 16; and

FIG. 18 is a schematic block diagram showing a plurality of modules that provide functionality to the patient-facing application shown in FIG. 16.

### DETAILED DESCRIPTION

The present disclosure relates to improved data structures for storing medical knowledge for use in diagnostic tools. In particular, the disclosure herein relates to a KG having a three-layered structure where medical knowledge is stratified into sub-KGs according to different medical “findings”, “interpretation” and “explanation layers”. Each finding is the root node of a sub-KG related to that particular medical finding. As described in detail below, this leads to a highly scalable solution that is capable of acting as a true “Knowledge Graph of Medicine” representing the state-of-the-art medical knowledge at any given time.

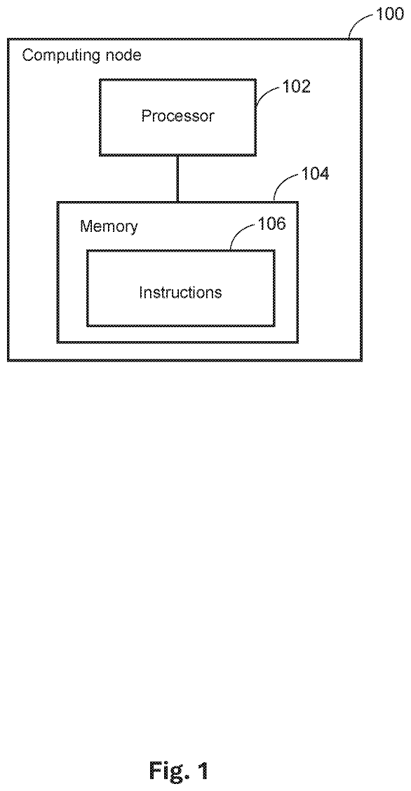

Embodiments of the disclosure herein are generally computer implemented and are performed by computer nodes. FIG. 1 shows a node (e.g. a computing node) that may form part of some embodiments herein. A node 100 may generally be configured (e.g. operative) to perform any of the methods and functions described herein, such as the methods 200, 800, 900 and 1300 described below. A node 100 comprises a processor 102, a memory 104 and set of instructions 106. The memory holds instruction data (e.g. such as compiled code) representing set of instructions 106. The processor may be configured to communicate with the memory and to execute the set of instructions. The set of instructions, when executed by the processor, may cause the processor to perform any of the methods herein, such as the method 200 described below.

Processor (e.g. processing circuitry or logic) 102 may be any type of processor, such as, for example, a central processing unit (CPU), a Graphics Processing Unit (GPU), a Neural Processing Unit (NPU), or any other type of processing unit. Processor 102 may comprise one or more sub-processors, processing units, multi-core processors or modules that are configured to work together in a distributed manner to control the node in the manner described herein.

The node 100 may comprise a memory 104. In some embodiments, the memory 104 of the node 100 can be configured to store program code or instructions that can be executed by the processor 102 of the node 100 to perform the functionality described herein. The memory 104 of the node 100, may be configured to store any data or information referred to herein, such as for example, requests, resources, information, data, signals, or similar that are described herein. The processor 102 of the node 100 may be configured to control the memory 104 of the node 100 to store such information.

In some embodiments, the node 100 may be a virtual node, e.g. such as a virtual machine or any other containerised computer node. In such embodiments, the processor 102 and the memory 104 may be portions of larger processing and memory resources respectively.

It will be appreciated that a computing node 100 may comprise other components to those illustrated in FIG. 1. For example, node 100 may comprise a power supply (e.g. mains or battery power supply). The node 100 may further comprise a wireless transmitter and/or wireless receiver to communicate wirelessly with other computing nodes. In some embodiments, the node 100 may have a wired connection with which to communicate with other computing nodes. In some embodiments, the node 100 may further comprise a user input device such as a mouse, keyboard, or touch pad, for receiving input user data. In some embodiments, the node 100 may further comprise a display for displaying any of the data described herein, such as for example, any of the outputs (or intermediate data products) of any of the methods described herein.

It will further be appreciated that the computing node 100 may form part of a wider system, such as a medical healthcare system. As such, the node 100 may be accessible via such a medical healthcare system to healthcare professionals, medical researchers and/or any other interested parties.

Generally, the processor may be configured to perform any of the methods described herein, such as the method 200 described below.

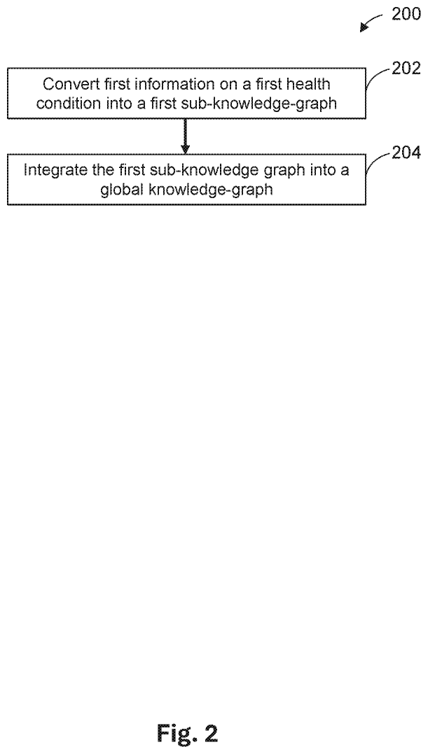

FIG. 2 shows a computer implemented method 200 for converting medical data into a graph structure for improved diagnostic querying according to some embodiments of the present disclosure.

In brief, the method 200 comprises converting 202 first information on a first health condition into a first sub-knowledge-graph, sub-KG. The first sub-KG has the following structure: a first layer comprising a first root node that is activated based on a first health indicator related to the first health condition; a second layer of nodes wherein each node in the second layer of nodes indicates a criteria to be applied to the first health indicator indicated in the root node; and a third layer of nodes, each node in the third layer of nodes being linked to a respective node in the second layer of nodes and corresponding to an interpretation associated with the first health indicator satisfying the criteria of the respective second layer node to which it is linked. In a second step 204 the method 200 then comprises integrating the first sub-KG into a global knowledge-graph (GKG). The GKG has a global root node and a first layer of nodes linked to the global root node. The step of integrating the first sub-KG into the GKG comprises adding the root node of the first sub-KG to the first layer of the GKG.

The method 200 can be performed by a computer such as the computing node 100 described in relation to FIG. 1 above.

In the first step 202, first information on a first health condition is converted into a first sub-knowledge-graph, sub-KG. The first health condition may be e.g. a disease (e.g. such as diabetes, high blood-pressure, heart failure, or any other disease), an illness (e.g. such as a virus or bacterial infection), a syndrome, or any other medical condition.

The first information may comprise a clinically validated set of data related to the first medical condition. The first information may have been obtained or stored in a “knowledge bank” (e.g. a database of medical information). The first information may have been derived from a variety of sources including, but not limited to, a textbook, a blog or other online resources, an article in an academic journal, or any other information related to the first medical condition. Generally, in embodiments herein, the first information may be in any form e.g. such as text-based information, audio information, photographic information, or any other format in which medical information might be presented. The first information may be structured or unstructured information. An example of information that may be used to create a first sub-graph for the medical condition “diabetes” is shown in Appendix A.

In step 202, the first information is used to generate a first sub-KG. As used herein, a sub-KG is a small KG relating to a particular health condition.

The skilled person will be familiar with knowledge graphs which are described in the following references: Hogan A., et al. (2021). Knowledge Graphs. ACM Comput. Surv. 54, 4. doi. org/10.1145/3447772 and Singhal A. (2012): “Introducing the Knowledge Graph: things, not strings.” Google Blog. blog.google/products/search/introducing-knowledge-graph-things-not. KGs have a graph structure that represents a network of real-world entities, wherein the information displayed is generally stored in a graph database. Generally, KGs comprise one or more nodes, edges and labels. Each node represents a datum comprised in the graph database and the nodes are connected by edges wherein an edge defines the relationship between pairs of nodes.

In the present disclosure, generally each node in the sub-KGs (and the global KG into which the sub-KGs are integrated) may comprise one or more symbolic rules wherein each rule comprises criteria that determine the relationships between each node and other nodes via the edges.

The sub-KGs herein have a particular structure. In a first layer, the first sub-KG has a root node, otherwise known as an input node. There is one input node in each sub-KG and thus the first layer of each sub-KG has a single node therein. The first layer may be referred to herein as a “finding layer” because the root nodes of the sub-KGs relate to (e.g. indicate) a particular clinical finding.

The first root node is activated (or activatable when in use) based on a first health indicator related to a first health condition. The first health indicator may be, for example, clinical finding such as e.g. a test (e.g. a physical test, or lab test) that can be performed on the patient. As such the root node of each sub-KG may relate to a different lab test result, relating to a different disease. Note that in use, the root node is activated if the patient data contains the first health indicator, e.g. the test result or other information, specified in the respective root node. In an example where the first health condition is diabetes, the first root node may relate to a Haemoglobin H1C measurement. In such an example, the root node may be activated if a Haemoglobin H1C measurement is available in the patient data.

In other examples, other health indicators may be used. For example, health indicators related to the environment, or geographic location in which the person lives. Examples of environmental health indicators include but are not limited to: air quality, water quality, pollutant levels, sun level, average pollen count, or any other environmental factor that may affect health.

In other examples, the health indicators may relate to risk factors such as whether the patient smokes, drinks alcohol, or exercises. As an example, the first root node may relate to the Carbon Monoxide level measured in the patient's breath (indicative of cigarette smoke inhalation). The skilled person will appreciate that these are merely examples however, and that a great many possible health indicators may form root nodes of the sub-KGs herein, for example, such as genetic or social determinants of health.

The sub-KG then has a second layer of nodes. Each node in the second layer of nodes is connected to the root node of the sub-KG via a respective edge. (E.g. the second layer of nodes radiates from the root node).

Each edge, for example, may specify how to process the information stated in the first root node of the first sub-KG to determine whether to actuate the node in the second layer to which the edge is joined. As an example, the edge may specify: “has_result_value” or: “is_present”. Each node in the second layer of nodes then indicates a criteria that can be applied to the value of the first health indicator indicated in the root node. As such, the edges linking the root node to the nodes in the second layer specify the type of criteria that is to be fulfilled for the adjoining node in the second layer to be activated. The nodes in the second layer specify the criteria that are to be used.

The second layer of nodes may be referred to as an Interpretation layer. The nodes in the interpretation layer set one or more criteria (e.g. define “a state”) that if satisfied, lead to a particular interpretation.

The first sub-KG then has a third sub-layer of nodes, which may be referred to herein as the “Explanation Layer” of nodes. Each node in the third sub-layer is joined to a respective node in the second layer via an edge. Edges between nodes in the second and third layers may indicate what to do when the second layer node to which it is joined is activated e.g. edges between the second and third layers of nodes may contain the instruction “interpret_as”.

The nodes in the third layer may then provide different interpretations for the results in the second layer.

An example path through a sub-KG for Diabetes is as follows:
Root Node: Haemoglobin A1c Lab Test;
Edge Between Root Node and Second Layer Node: has_result_value”;
Adjoining Second Layer Node: “between 4.0% and 5.7%”;
Edge between Second Layer Node and Third Layer Node: “interpret_as”;
Adjoining Third Layer Node: “Normoglycemia”.

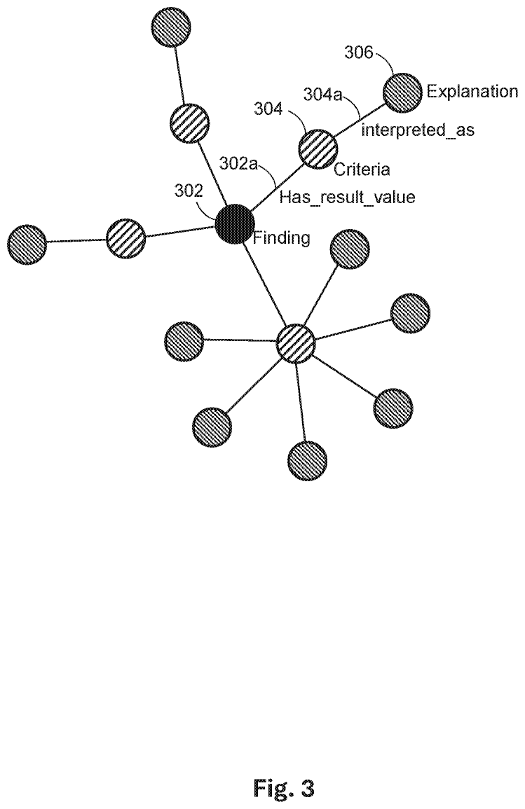

An example first sub-KG is illustrated in FIG. 3 which shows the structure described above. The example first sub-KG has a root node 302 which represents a “finding” e.g. “a clinical finding” for the patient such as a clinical test result or health marker that may be present in a patient's medical record. A plurality of edges 302a link the root node 302 to nodes in the second layer indicated by the thick hatched circles 304. Edges 302a describe how the finding or data specified in the root node 302 is to be assessed e.g “Has_result_value”. Nodes in the second layer (interpretation layer) then stipulate a criteria such as a range of values that if satisfied, activate node 304. The nodes in the interpretation layer provide different criteria for different interpretations of the patient's clinical findings. In use, the patient's clinical findings are interpreted and the results are housed in the Interpretation nodes. Nodes in the second layer are linked to nodes in the third layer indicated by the fine hatching 306 by edges 304a. Nodes in the third layer provide an explanation for the “finding” in node 302 having a result value satisfying the “criteria” of the node 304. It will be appreciated that the number and arrangement of nodes indicated in FIG. 3 is merely an example, and the first sub-KG may have any number of nodes and edges in the second and third layers.

Generally, the first, second and third layers may be uni-directional. In this sense, there are no reverse edges between the nodes in the first layer (e.g. the first root node), the nodes in the second later (interpretation nodes) and the nodes in the third layer (explanation nodes). This simplifies the structure, allowing for uniformity in the presentation and storage of the data and enables the sub-KGs to be queried in parallel with one another leading to quick query times.

In some embodiments, there may be further layers. For example, the first sub-KG may further comprise one or more further layers. The nodes in the one or more further layers may represent different explanations for each interpretation of a respective third layer node. This captures the fact that e.g. a particular test result in a particular (abnormal) range may have more than one explanation or cause. Nodes in the further explanation layers may be bi-directional, e.g, with reverse edges.

Explanation nodes that are common to more than one sub-KG may be linked e.g. using a Machine Learning Model that looks for semantic similarities between nodes in the third layer and links common explanation nodes into a single node. The links between the explanation nodes may be bi-directional (e.g. there may be reverse edges present). The explanation nodes can also be linked by parsing standard Medical Ontologies like SNOMED and the Unified Medical Language System (UMLS), comparing terminologies and detection the concepts semantic similarity. SNOMED is described in the document: “SNOMED CT Managed Service US Edition SNOMED CT to ICD-10-CM Release Notes—March 2024” dated 2024 Mar. 1 and available at: https://confluence.ihtsdotools.org/display/DOC. UMLS is is described in the following paper: Bodenreider O. The Unified Medical Language System (UMLS): integrating biomedical terminology. Nucleic Acids Res. 2004 Jan. 1; 32(Database issue): D267-70. doi:10.1093/nar/gkh061. PubMed PMID: 14681409; PubMed Central PMCID: PMC308795.

The first KG may be generated from the first information in different ways. For example, the skilled person will be familiar with methods of manually creating a KG from information related to a disease indicator or marker.

In some examples herein a large language model (LLM) is used to convert the first information into the first sub-KG. For example, a prompt may be sent to an LLM, along with the first information, that instructs the LM to create a KG, from the first information, in the structure specified above. There are significant advantages associated with the use of LLMs to generate the sub-KGs. In particular, the sub-KGs can be created in an automatable manner, without the need for human input through the use of LLMs. This significantly reduces the amount of work need to create each sub-KG meaning that considerably more sources of information can be incorporated into the global-KG, furthering the goal of producing a global-KG of medicine. Furthermore, the sub-KGs can be created in a uniform manner using uniform terminology in this way. Embodiments where LLMs are used to create the sub-KGs are described in more detail below.

In step 204 of the method 200, the first KG is integrated into a global-KG. The global KG comprises a root node and a plurality of sub-KGs, each of the sub-KGs having the same structure as the first sub-KG described above. The first sub-KG is integrated into the global-KG by adding the root node of the first sub-KG into the first layer of the global KG.

In some embodiments, the sub-KGs are stored as separate data sets which can be queried (e.g. pinged or activated) from the global KG. The sub-KGs are queried based on a series of “if-then” statements which may be written by an artificial intelligence (AI) algorithm. For example, a statement may comprise the following rule:

If has_result_value=‘Hemoglobin A1c Lab Test’ then query ‘Hemoglobin A1c’ node (e.g. the ‘Hemoglobin A1c’ sub-KG).

These statements are also discriminatory to prevent hallucinations and to prevent incorrect nodes from being queried. As such, in some embodiments, in step 204 the first KG is integrated into the global-KG by means of a new if-then statement (equivalent to a new edge from the global-root node) being added to the global-KG, that indicates (from the global root node) when the respective sub-KG is to be activated.

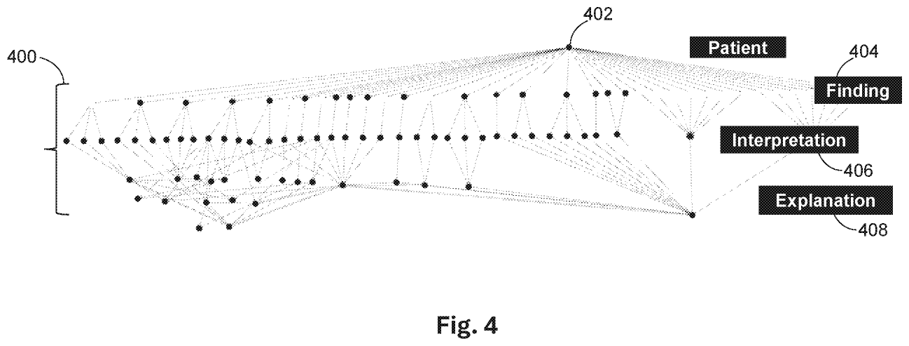

The global-KG has the structure illustrated in FIG. 4 which shows a global-KG 400 having a root node 402, a first layer comprising the “finding” or root nodes 404 of a plurality of sub-KGs (each created and integrated into the global-KG using the method 200), a second layer 406 comprising the interpretation nodes of all of the sub-KGs and a third layer 408 comprising the explanation nodes of the sub-KGs. It is noted that more than one interpretation node in more than one sub-KG may be linked to a particular explanation node as there may be more than one test that can be used to make a particular diagnosis. As noted above, the explanation nodes can also be linked by parsing standard Medical Ontologies like SNOMED and UMLS, comparing terminologies and through the detection of semantic similarity between concepts in the explanation layer.

The method 200 may be repeated, for example, to convert second information on a second health condition into a second sub-KG, the second sub-KG having the same structure as the first sub-KG. The second sub-KG may be integrated into the global-KG, by adding the root node of the second sub-KG to the first layer of the global-KG.

This process may be repeated using a plurality of information sources and a plurality of health conditions in order to build up a comprehensive KG of a wide-range of health conditions. In this sense, the global KG may thus be considered a Knowledge Graph of Medicine.

The global-KG may also be referred to herein as a Condition Dynamic Digital Twin (CDDT) or a Disease Digital Twin (DDT). It is a structured representation of vast amounts of medical knowledge that captures relationships between various entities such as diseases, symptoms, drugs, and treatments. The global KG is organised into a graph structure, as described above. The global KG structure advantageously captures the complex connections between medical concepts and enables computationally efficient navigation of the graph to retrieve information.

The global-KG herein is formed of a plurality of sub-KGs as described above, this makes the solution scalable as there is no limit on the number of sub-KGs that can be incorporated into the larger global-KG.

Furthermore, because the sub-KGs are largely isolated from each other, they can be individually updated, separately to one another. This simplifies the process of updating the global-KG when new/updated information is obtained.

Turning now to another embodiment, as briefly noted above, in some embodiments herein, the sub-KGs herein may be created in step 202 using LLMs. For example, in some embodiments, step 202 may comprise prompting a large language model, LLM, to convert the first information into the first sub-KG. In such embodiments, the prompt used provides the first information and a description of the structure that the first sub-KG should take to the LLM.

Examples of LLMs that can be used to convert the first information into the first sub-KG include but are not limited to: GPT-4-32k (OpenAI (2023) “GPT-4 Technical Report”. arxiv.org/abs/2303.08774), Llama-2-70b-chat (GenAI, Meta (2023). “Llama 2: OpenFoundation and Fine-Tuned Chat Models”. arxiv.org/abs/2307.09288) and Mistral-large (MistralAI (2024) version 24.07 [Large language model]).

In some embodiments, a general purpose LLM, such as those cited above can be “tuned” using techniques such as directional stimulus prompting (DSP) which is described in the paper by Li Z., et al. (2023) entitled: “Guiding Large Language Models via Directional Stimulus Prompting”; arxiv.org/abs/2302.11520. Retrieval Augmented Generation (RAG) or a combination of RAG with DSP can also be used.

DSP is a prompt technique which involves generating and providing the LLM with nuanced, instance-specific directional stimuli (i.e. hints) to guide the LLM in generating the desired outcome or response. In more detail, the directional stimulus prompt acts as a “hint” or “clue” for the input query to guide the LLM toward the desired output. This technique uses a tuneable policy language model (LM) that is trained to generate the stimulus/hint. Advantageously, training the policy model avoids direct optimisation of a black-box LLM and enables a small tuneable policy model to be optimised instead.

RAG is a process used to optimise the output of a LLM, by grounding the LLM on an authoritative knowledge base outside of its training data sources before generating a response. This process aims to overcome some of the shortcomings of LLMs, such as producing “hallucinations” when presented with queries beyond their training data or requiring current information. The RAG approach redirects the LLM to retrieve relevant information from reliable, pre-determined knowledge sources without needing to retrain the LLM. Therefore, grounding the LLM on a set of external, verifiable facts reduces the probability of the LLM hallucinating incorrect or misleading information. In particular, the knowledge base is used to create text embeddings for the RAG-based techniques. Additionally, the knowledge sources can be automatically updated to maintain up to date information. As such, the RAG technique ensures that the LLM has access to the most current and reliable facts, and that the users of the LLM have access to the knowledge sources, ensuring that the LLM output can be checked for accuracy and ultimately trusted. This is particularly advantageous in the context of medicine, as false information can endanger human lives and lead to mistrust of the underlying AI technology.

RAG is described in the following academic papers: D. Arora, A. Kini, S. R. Chowdhury, N. Natarajan, G. Sinha, and A. Sharma, “Gar-meets-rag paradigm for zero-shot information retrieval,” arXiv preprint arXiv: 2310.20158, 2023. P. Lewis, E. Perez, A. Piktus, F. Petroni, V. Karpukhin, N. Goyal, H. K\ddot{ }uttler, M. Lewis, W.-t. Yih, T. Rockt\ddot{ }aschel et al., “Retrievalaugmented generation for knowledge-intensive nlp tasks,” Advances in Neural Information Processing Systems, vol. 33, pp. 9459-9474, 2020. S. Borgeaud, A. Mensch, J. Hoffmann, T. Cai, E. Rutherford, K. Millican, G. B. Van Den Driessche, J.-B. Lespiau, B. Damoc, A. Clark et al., “Improving language models by retrieving from trillions of tokens,” in International conference on machine learning. PMLR, 2022, pp. 2206-2240.

The combination of RAG with DSP incorporates a retriever component e.g. referencing information such as a knowledge base to retrieve specific information, with an augmented generation step in which the LLM prompt incorporates both the retrieved information as well as a directional stimulus (or hint) in the prompt.

The generation of the sub-KGs in step 204 will be further explained below with reference to the following examples.

### First Example of Generating a Sub-KG for Diabetes

In this first example, a sub-KG is built for the illness diabetes. Diabetes is indicated by the average blood glucose level of a patient that can be measured in a HbA1c test. The HbA1c test is therefore comprised in the “finding” layer of the sub-KG.

To build a sub-KG around the Hb1Ac result, a text input (prompt) is provided to an LLM. In this example, the Llama-2-70b-chat LLM is used, implemented with a combined RAG with DSP approach as described above. The information input to the LLM is given in Appendix A.

As an example, an LLM prompt can be formulated as follows:

Extract the following information regarding Hemoglobin A1C (Hba1c):
(A) A numbered list of the different lab test result ranges for the Hemoglobin A1c Lab Test

Please be comprehensive and do not miss any relevant information.

A more detailed prompt can be determined using the DSP technique described above. An example prompt derived using DSP is given in the following text:

Create a knowledge graph, consisting of Nodes and Relationships.
nodes: Each node has a unique id, a caption, labels, and properties. Here are the main fields for each node:
   id: A unique identifier for each node. It should include the caption of the node
   caption: A name that will be displayed on the node.
   labels: Defines the type of node, so nodes can be grouped into those of the same type.

There are a limited set of options for a node's label.
properties: A dictionary of additional properties for the node, if relevant.

The options for a node's label is limited to the following set (in no particular order):
1. LabTest: A specific Lab Test which can be conducted on a patient. This node serves as the starting node of this graph.
2. LabTestValueRange: The result or value obtained from a specific Lab Test.
3. Condition: The medical Condition by which you can interpret a given LabTestValueRange.
4. Dx: A clinical diagnosis.
5. Finding: A clinical finding or observation.

The label will have one of the above 5 terms as a list, for example: [“LabTest”].

relationships: A relationship represents a directed, conceptual link between two nodes. Each relationship has a unique id, a start node id (fromId), an end node id (toId), a type, and properties. Here are the main fields in the relationship data model:
id: A unique identifier for each relationship.
fromId: The id of the node where the relationship starts.
toId: The id of the node where the relationship ends.
type: Defines the type of relationship between the two nodes, so relationships can be grouped into those of the same type. There are a limited set of options for a relationship's type.

The options for a relationship's type is limited to the following set (in no particular order):
1. has_result_value: Connects a LabTest node to a LabTestValueRange node.
2. interpreted_as: Connects a LabTestValueRange node to a Condition node.
3. has_cause: Connects either (A) a Condition node to a Finding or Dx node; or (B) a Dx node to a Dx node.

Use the Graph Data Model described above and produce a Knowledge Graph for Hemoglobin A1C (HbA1c). All nodes in the graph should have at least one relationship to another node; there should be no floating nodes. All potential results of the lab test should be reflected and traced back to an interpretation, then potential causes, if applicable. Utilize the following knowledge base to populate the Hba1c graph.

The “knowledge base” referred to in this example prompt corresponds to the first information described above with reference to FIG. 2 and comprises clinically validated set of data about the health condition. As noted above, an example excerpt from the knowledge base (e.g. the “information” that can be input to the LLM) to construct the sub-KG for HbA1c is shown in the text in Appendix A.

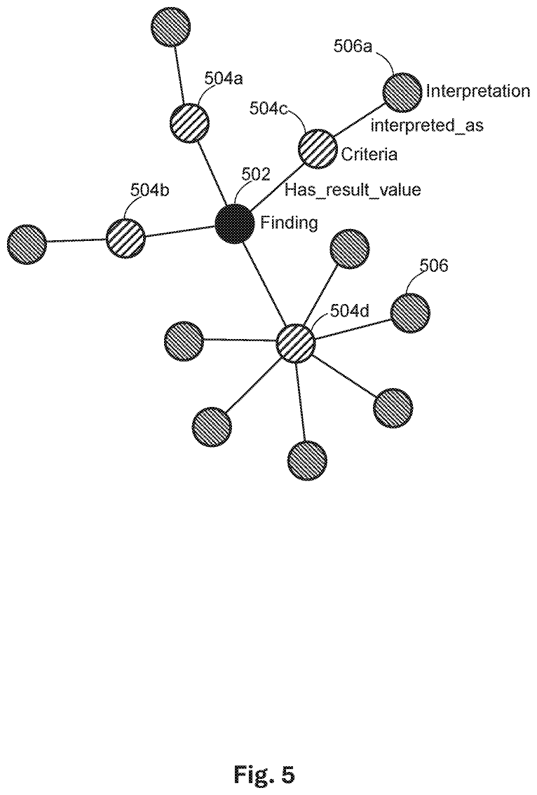

The output of the LLM using the prompt and information in Appendix A in the First Example above is illustrated in FIG. 5. This sub-KG comprises a central finding node 502 labelled “Hemoglobin A1C” which branches out to a plurality of interpretation nodes via edges comprising criteria in the form of four criteria nodes 504a-c. These criteria nodes comprise the following symbolic rules determined from the input data: a value of less than 4% 504a; a value of between 4.0% and 5.7% 504b; a value of between 5.7% and 6.5% 504c; and a value greater than 6.5% 504d. Each of these criteria nodes connect the finding node 502 to nodes comprised in the interpretation layer through edges labelled as “interpreted_as”. For example, the criterion node of a value of between 5.7% and 6.5% 504c branches out to an interpretation node labelled as “Prediabetes” 506a. It is possible to have multiple possible interpretations for a test result which is exemplified by the criterion node representing a value of greater than 6.5% 504d branching out to a plurality of interpretation nodes 506.

### Second Example of Generating a Sub-KG for Diabetes

The process for generating the sub-KG shown in FIG. 5 according to the First Example Embodiment can equally be carried out with a different LLM model and approach. As a Second Example, the GPT-4-32k LLM model may be used with a combined RAG with DSP approach.

An example of the query input to the LLM (again based on the example input information or knowledge base illustrated in Appendix A) is as follows:

Extract the following information regarding Hemoglobin A1C (Hba1c):
(A) A numbered list of the different lab test result ranges for the Hemoglobin A1c Lab Test
(B) For each Hemoglobin A1c Lab Test result range in (A), the corresponding interpretation.
(C) For each interpretation in (B), a numbered list of the various causes (e.g., Findings, Diagnoses or Dxs, etc.) for that interpretation.
(D) Where applicable, for Findings and Diagnoses returned as causes in (C), additional downstream causes.

Please be comprehensive and do not miss any relevant information.

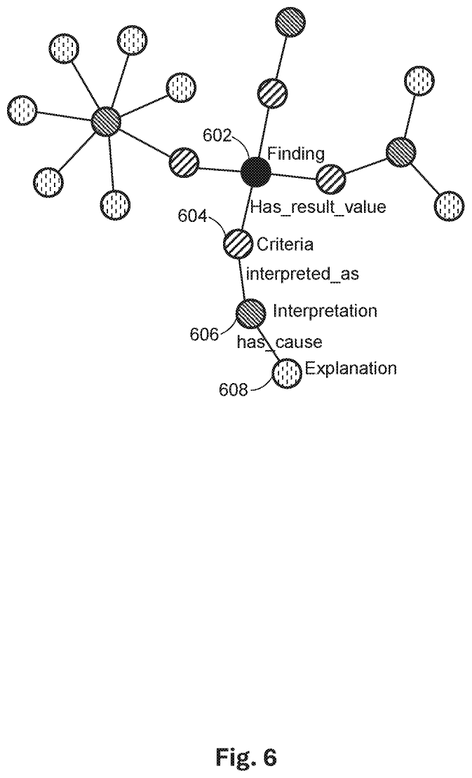

The output of the LLM in the Second Example is shown in FIG. 6. This sub-KG comprises a central “finding” node 602, which branches out to four interpretation nodes via edges comprising criteria in the form of four interpretation nodes 604. These interpretation nodes 604 comprise the symbolic rules/criteria from the input data, wherein the rules comprise the different ranges of the Hb1Ac values outlined above. The “interpreted_as” edges therefore essentially connect the finding node to the “interpretation” layer 606 via the criteria nodes 604. The interpretation layer 606 is in turn connected to the “explanation” 608 layer by edges labelled as “has_cause”. For example, an Hb1Ac value of less than 4.0% is interpreted as “Sustained Hypoglycemia” which can be explained by two different causes: extreme diet restriction or excessive use of antidiabetic drugs. In other embodiments, the number of explanations may be fewer or greater than two and may be zero in embodiments where the “finding” value falls within a clinically normal range.

Each sub-KG generated by the LLM can be validated by a clinician to ensure clinical accuracy of the sub-KG. Once a sub-KG is validated by a clinician, the output is referred to herein as a clinically validated sub-KG.

In some embodiments, the sub-KGs can be validated in an automated manner by comparing the sub-KGs to a gold set (e.g. a ground-truth set) of sub-KGs for different diseases. The gold set may be made up of a manually curated sub-KGs generated by clinicians that represents the “ideal” sub-KG for a particular information source as would be generated manually by a clinician. A similarity scoring system may output an accuracy score for each sub-KG.

The comparison may be carried out using quantitative or qualitative methods and is used to assess the best performing LLM as further described below.

Qualitative methods of comparison may comprise evaluating the outputs based on the following criteria:
1. Factual Accuracy of KG components: evaluation of the clinically accuracy of the medical concepts output by the model;
2. Structural similarity of KG: evaluation of the structural similarity between the KG and the gold set; and
3. Comprehensiveness of KG: evaluation of the degree of completeness of the KG i.e. whether all clinical concepts in the gold set are also present in the KG.

Quantitative methods of assessing the output of an LLM may be based on purpose-built graph similarity scores. The scoring system assigns a score to the LLM based on the comparison between the KG and the gold set, and determines the “best” LLM, e.g. the LLM that generates the most accurate KG based on the similarity of structure between the KG and the gold set and the extent of data capture from the input information, based on the score.

Thus, in this way, validation can be performed in an automated manner, which can be used to update the global-KG upon receipt of new or updated clinical information.

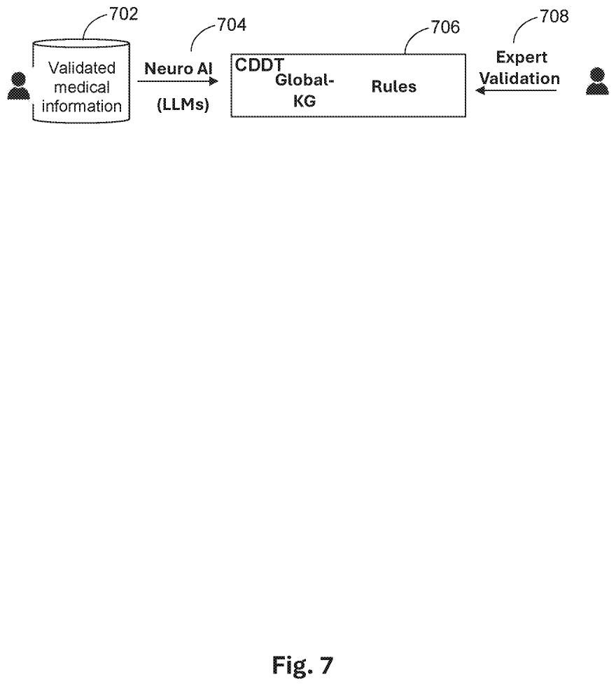

Turning now to FIG. 7, which provides a summary of some of the components that may be involved in creating a global-KGs according to some embodiments herein. As described above, information comprising validated medical knowledge in text format (or any of the other formats described above) is input from a database 702 into a large language model (LLM) 704. This information may comprise first information on a first disease and is processed by the LLM to generate a first KG or sub-KG corresponding to the first disease. By inputting a plurality of information relating to a plurality of diseases into the LLM, a plurality of sub-KGs can be generated wherein each sub-KG corresponds to a different disease. The plurality of sub-KG can then be embedded into a larger KG (the global-KG described above). Accordingly, the global KG 706 or CDDT or DDT comprises one or more sub-KGs, linked together by rules. As described above, each sub-KG and therefore the global KG can be clinically validated by a clinical expert 708. Alternatively, the accuracy of a sub-KG can be automatically assessed using methods such as purpose-built graph similarity scores described above.

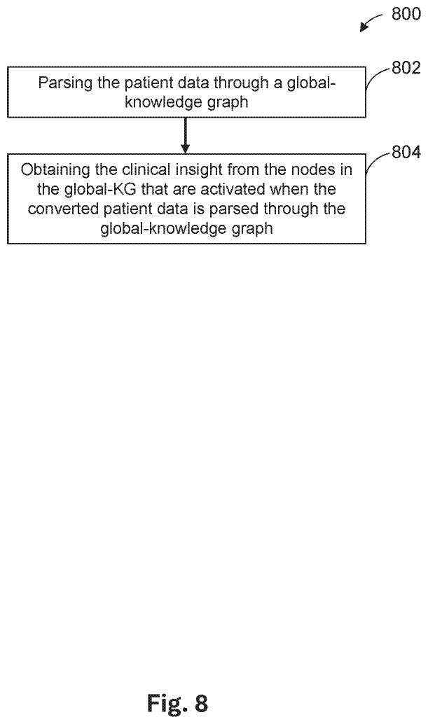

Once created, the global KG can then be used to diagnose medical condition(s) in a thorough and robust manner. FIG. 8 shows a computer implemented method 800 of processing patient data to obtain a clinical insight according to some embodiments herein. In this sense, a clinical insight may be a diagnosis, an indication of a possible predisposition, or any other insight into the patient's health.

The method 800 can be performed on a computer such as the computer node 100 of FIG. 1. In brief, in a first step 802, the method 800 comprises: i) parsing the patient data through a global-knowledge graph (GKG), the GKG comprising a plurality of sub-knowledge graphs (sub-KGs), wherein each sub-KG has the following structure: a first layer comprising a first root node that is activated based on a first health indicator related to a first health condition; a second layer of nodes wherein each node in the second layer of nodes indicates a criteria to be applied to the first health indicator indicated in the root node; and a third layer of nodes, each node in the third layer of nodes being linked to a respective node in the second layer of nodes and corresponding to an interpretation associated with the first health indicator satisfying the criteria of the respective second layer node to which it is linked. The GKG has a global root node and a first layer of nodes linked to the global root node, and wherein the plurality of sub-KGs are integrated into the GKG by means of their respective root nodes having been added to the first layer of the GKG. In a second step, 804, the method 800 comprises: ii) obtaining the clinical insight from the nodes in the GKG that are activated when the converted patient data is parsed through the GKG.

In other words, the patient data is parsed through a GKG with the structure described above with respect to the method 200 in FIG. 2. The patient data may be pre-processed. For example, preceding steps 802 and 804 above, the method 800 may comprise converting the patient data into a structured data format. For example, if the patient data is unstructured (e.g. such as in a written or audio recorded format), then the patient data may be converted into a structured data format before being parsed through the GKG. In some embodiments herein, step 802 may be performed by an artificial neural network (ANN).

At Step 802, the structured data is parsed through (e.g. input to) the global root node of the GKG. As described above, with respect to the method 200, the global root node, also referred to herein as the “patient node”, is comprised at the top of the graph structure of the GKG. The global root node branches out to one or more nodes in the first layer of the global KG. Various examples of the graph structure of the sub-KGs and the GKG were described above with respect to FIGS. 3 to 6.

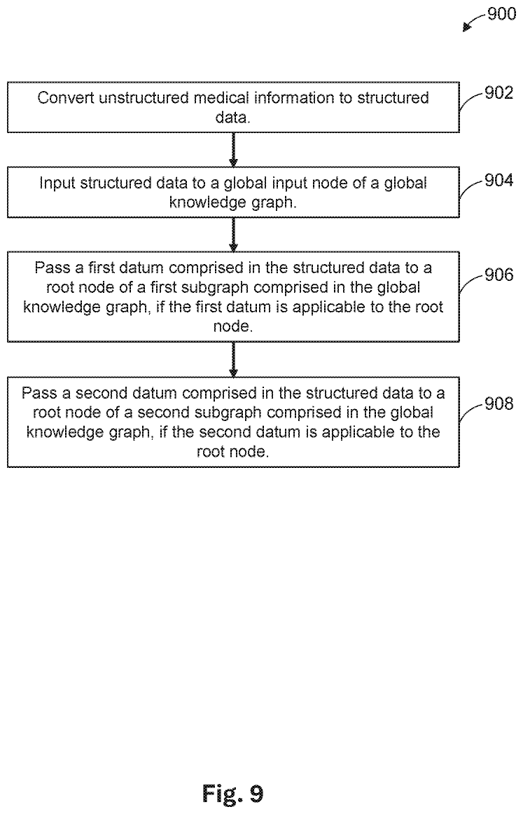

An embodiment illustrating example sub-steps that may be performed in step 802 are shown in FIG. 9. In FIG. 9, a first step 902 comprises converting the unstructured medical data into structured data, as described above. The structured data is then input 904 to a global input node of the global-KG. The patient data is then passed on a datum-by datum basis through the global-KG. Each datum (e.g. piece of data/information in the patient data) is compared to the root nodes of the sub-KGs in the GKG and is subsequently passed through the respective sub-KG if it is applicable to the respective root node.

For example, a first datum comprised in the structured patient data may be passed, at Step 906, to a first root node of a first sub-KG comprised in the global KG, if the first datum is applicable to said first root node. For example, if the first datum comprises an Hb1Ac test result value, the datum is passed to the appropriate Hb1Ac test result node comprised in the first layer (or finding layer) of the global-KG.

Similarly, a second datum comprised in the structured data may be passed, at Step 908, to a root node of a second sub-KG comprised in the global knowledge graph, if the second datum is applicable to (e.g. satisfies the criteria of) the root node.

As described above, in some embodiments each sub-KG is stored separately and can be queried based on a series of if-then style statements from the global root node of the global-KG. Thus, if a datum is applicable to a sub-KG (e.g. if the root node of said sub-KG is activated by the datum), then this may trigger the respective sub-KG to be loaded into memory.

These steps are repeated until each relevant datum comprised in the patient data has been input to an appropriate root node or nodes of one or more sub-KGs.

The datums of patient data subsequently flow (e.g. are passed) through the respective sub-KGs according to the rules and criteria comprised in the nodes and edges.

If the patient data satisfies one or more diagnostic criteria comprised in a node or edge, the data traverses to one or more nodes in a subsequent layer of the global KG. Each node or edge successfully traversed by the patient data becomes “activated”. Nodes that are activated as the patient data flows through them may be flagged as such (e.g. via a mechanism such as setting an “activation” Boolean associated with each node). As such, activated nodes represent nodes in the sub-KGs (and thus the global-KG overall) that are relevant to the patient in some way.

In step 804, the clinical insight is obtained (or determined) from the activated nodes. For example, the clinical insight may be obtained from the activated nodes in the “Explanation” layer of the global-KG and associated sub-KGs, which may provide explanations for various test results and the like.

In some embodiments, a patient-specific KG may be created, comprising just the nodes of the global-KG that are activated when the patient data is fed through it. A patient specific KG can be obtained by creating a copy of the global-KG with nodes and edges that are not activated by the patient's data having been “pruned” away e.g. deleted or removed. Such pruning can be performed by comparing the activated nodes and edges to the global KG. The activated nodes and edges remaining after pruning are referred to herein as the patient-specific-KG or the “Computational Health Graph” (CHG). The patient-specific KG comprises the activated nodes and edges of the global KG that can be used to explain the medical condition(s) of the respective patient. The patient-specific KG acts simultaneously as both a representation and explanation of the medical data for the particular patient.

Advantageously, the underlying structure of the global KG ensures that the flow of information through the global KG is performed in a methodical and robust manner, providing a comparison with every disease/health condition/health risk factor stored in a sub-KG therein. Furthermore, as described in more detail below, the determination is deterministic and fully repeatable resulting in the same output for a given input.

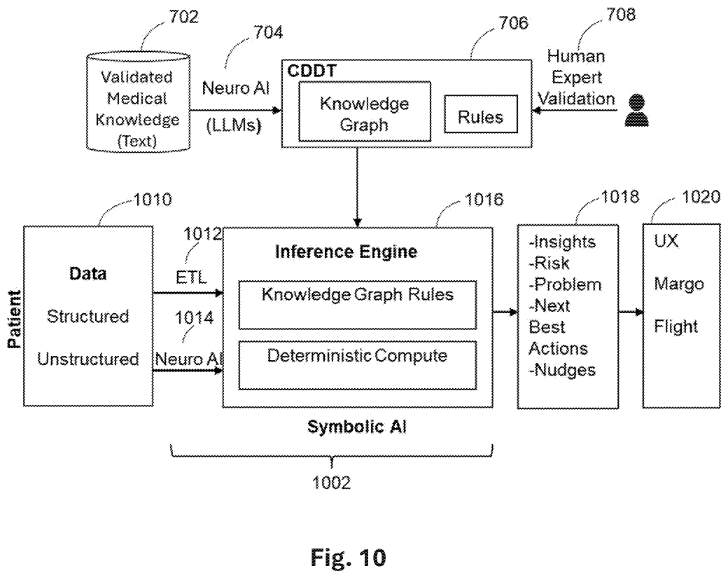

FIG. 10 shows a block diagram for creating a patient-specific-KG (or CHG) according to some embodiments herein. As described above, a global KG 706 may be generated using validated medical information in text format 702 that is processed by a LLM 704 to create one or more sub-KGs wherein each sub-KG corresponds to a different disease. The nodes and edges comprised in the global KG comprise rules and criteria that determine the structure of the global KG i.e. how the nodes are connected to each other.

As information flows from the Findings through the Interpretations and then successive layers of Explanations, rules embedded in the nodes and edges are executed by a Symbolic AI system to draw conclusions about the activation state, validity or invalidity, and the degree of support (support_score) for each explanation node. The support scores are in the model itself. The scoring is based on the frequency of events in the general population. The score is given as 0-1, existing or not existing e.g. supports or rejects. The number of times that a node is represented in the population gives the frequency. On an individual level, 1 means there is a risk factor, 0 means there is no risk factor. The risk factor can be e.g. genetic, environmental or clinical.

In some embodiments, the patient-specific-KG or CHG is generated by querying the general global KG using a machine learning (ML) model referred to herein as a “Dynamic Linking Engine” 1002. The Dynamic Linking Engine 1002 is based on neuro-symbolic artificial intelligence (AI). The skilled person will be familiar with neuro-symbolic AI as a type of AI that combines neural AI and symbolic AI. The Dynamic Linking Engine 1002 comprises a neural AI model 1014 e.g. an ANN, and an “Inference Engine” 1016 wherein the Inference Engine 1016 comprises a symbolic AI model.

To generate a CHG for a patient, data associated with the patient 1010 is input to the Dynamic Linking Engine 1002 and mapped onto the GKG 706. The patient data may comprise structured data from an electronic health record (EHR) system, unstructured data from e.g. a physician's notes, or both structured and unstructured data. The patient data may comprise one or more of: genetic data; environmental information; social determinants of health; and clinical test results (such as the Hb1Ac test result mentioned above). Additionally, the patient data may comprise a “patient 360 (P360)”, wherein the P360 comprises a comprehensive database of all patient-specific health data that is available for the patient in question. The P360 may comprise medical history, electronic health records (EHR), service history, claims data, patient feedback on their own health, and self-reported lifestyle and demographic data. Additionally, the patient data may comprise various formats e.g. text, hand-written notes, audio files or video files. Therefore, the patient data may comprise a plurality of data points wherein each data point corresponds to a different health indicator.

The patient data is converted to a structured format using the neural AI model comprised in the ML model, wherein the neural AI model essentially uses an Extract, Transform and Load (ETL) process 1012. The skilled person will be familiar with an ETL process 1012 as a process commonly used for data management. In embodiments herein, the ETL process 1012 used by the neural AI model converts the patient data into a structured text format.

The structured information and the global KG are then input to an Inference Engine 1016. The symbolic AI model comprised in the Inference Engine 1016 inputs the patient data to one or more root nodes (input nodes) comprised in the first layer (finding layer) of the global KG. The symbolic AI then uses symbolic reasoning to traverse the patient data from the one or more root nodes over the global KG. For example, the patient data may comprise a medical test result such as a Hb1Ac test result. Therefore, the Hb1Ac test result is input to the relevant root node and this root node becomes “activated”. A set of rules constrains the graph traversal wherein the rules are based on expert input e.g. a clinician's input. The rules are embedded in the inter-relations e.g. edges connecting the nodes of the global-KG.

Subsequently, the patient data from the one or more root nodes is traversed to one or more layers comprised in the global-KG. The route of traversal depends on the rules and criteria comprised in the nodes and edges of the global KG. The rules and criteria may comprise diagnostic criteria used to diagnose an aspect of a disease and the edges between the nodes link the respective diagnostic criteria into sequences of diagnostic criteria that if met, lead to particular diagnoses associates with the disease.

For example, the Hb1Ac test result comprises a value indicating the level of blood glucose of the associated patient. Depending on the value of the Hb1Ac test result, the rule or criterion comprised in one of the edges branching out from the root node is satisfied. This edge therefore becomes “activated” and the GKG is traversed to the node in the second layer e.g. interpretation layer, that is at the other end of this edge. As a result, the patient data or findings are interpreted, and the results are housed in the nodes comprised in the second layer e.g. interpretation nodes.

Similarly, traversing the GKG from the second layer to a third layer e.g. explanation layer, of the GKG, is dependent on satisfying the rules or criteria of the edges connecting the nodes comprised in the second layer to the nodes comprised in the third layer. As a result, the interpretations of the patient data, represented by the activated nodes in the interpretation layer, are reasoned over based on the relationships between the nodes as well as the rules or criteria embedded in the nodes and edges. The activated nodes in the explanation layer (referred to herein as explanation nodes) comprise an explanation for the associated patient's clinical state.

The nodes and edges which have been activated are subsequently compared with the global KG and the inactivated edges and nodes are pruned away i.e. removed from the knowledge graph. As noted above, the graph that remains after pruning comprises the activated nodes and edges and this graph is referred to herein as the patient-specific-KG or Computational Health Graph (CHG). Not only does it explain the patient's clinical “findings” but it shows its reasoning, making it an explainable AI solution. In this sense, it is a personalised KG. The explanation nodes comprised in the CHG can then be converted to a list of health problems or risk factors 1018, which can then be sent as an electronic message 1020 to the patient and/or clinician e.g. through a mobile application, a text message or an email.

To improve the accuracy of the CHG, the patient data input to the root node may be classified as “encounter data” or “non-encounter data”. Encounter data includes data collected during an interaction between a patient and a healthcare provider (e.g. a clinician). Encounter data may comprise vital sign data, physical examination findings, diagnostic test results, or lab data e.g. HBA1c levels collected during a clinical visit. Non-encounter data may include data from remote patient monitoring devices, such as a glucose monitoring device, data from a wearable device, or self-reported patient information collected remotely. Patient data can be flagged as “encounter data” to be processed by the symbolic AI model according to rules and criteria that apply to encounter data. The encounter data input to the GKG comprises a test result and the context of the test result e.g. the specific conditions under which lab tests or physical examinations were performed. The encounter data can often be helpful to establish the veracity of the patient data being input as it provides the context under which that encounter data was obtained which is important in the weighting that a clinician may give that data. The encounter data thus provides an additional dimension to the data in the CHG which is useful for clinicians.

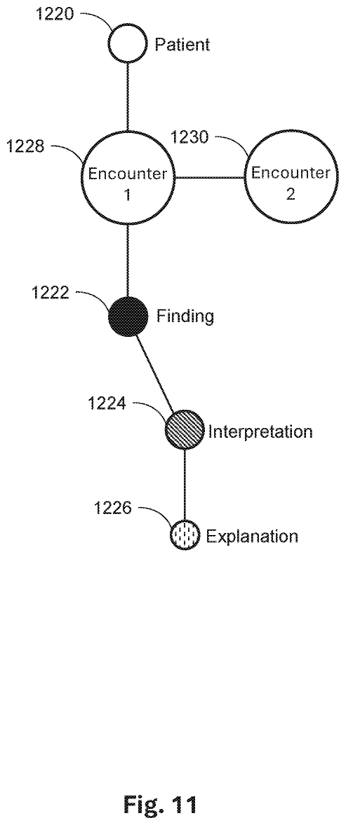

As shown in FIG. 11, encounter data input to the global root node or patient node 1220 of the GKG is input to a subsequent layer of nodes i.e. encounter nodes 1228, 1230, that are comprised in the graph structure of the GKG between the patient node 1220 and the finding layer 1222. During a clinical visit, the patient may have multiple “encounters” e.g., different lab tests or clinical tests, and each encounter datum is comprised in a separate node. Non-encounter patient data is also input to the global root node or patient node 1220 of the GKG. The non-encounter data and the encounter data can then be used to generate the patient-specific-KG or CHG according to method 800 described above with reference to FIG. 8 and FIG. 9.

Each additional encounter or non-encounter datum may result in an additional finding node being “activated” when the patient data is parsed through the GKG. The information relating to the context of a datum is traversed through the GKG and can be used to provide additional insight into the acute and chronic conditions managed during face-to-face interactions. The information that comprises the health indicator is traversed through the GKG according to the rules and criteria embedded in the nodes and edges as described above. The encounter data therefore affects the degree of support (support_score) for each explanation node in the same manner as non-encounter data.

Classifying patient data as encounter data and non-encounter data allows for a more detailed understanding of a patient's health, as both situational and ongoing health information are encompassed. Also, differentiating between encounter data and non-encounter data may help identify health patterns that may not be evident from isolated data points. For example, repeated high HBA1c levels during clinical visits can indicate poor diabetes management, necessitating a change in treatment strategy. Similarly, frequent visits for acute conditions might suggest underlying chronic issues that need addressing.

In some embodiments, the Dynamic Linking Engine 1002 may further comprise a machine learning (ML) model, herein referred to as the Health Dynamic Link Library, (HDLL) that is used to predict a level of care required by the patient. To predict a level of care, the CHG is input to the ML model and the ML model uses a series of rules to categorise the level of care required by the patient. For example, these categories may include wellness e.g. no health care required, easy care, complex care, or high level care.

The advantage of implementing symbolic AI to reason over the global KG e.g. to traverse the global KG with patient data, ensures that the graph is traversed (or queried) in a methodical and robust way, and leads to an explainable result.

Traversal of the global KG and “activation” of the appropriate nodes is performed using deterministic computation. Deterministic computation is advantageous for avoiding the randomness usually associated with ANNs and ensures that the same result is output each time the model is run (as long as no parameters are altered between model runs).

An advantage of the structure of the global KG is that it can be queried in parallel when the patient data input to the global input node comprises two or more data points. This results in fast and extremely computationally efficient graph traversal.

Moreover, each sub-KG of the global KG which is queried as a result of the root node being activated, is queried using a depth first search (DFS) algorithm. The skilled person will be familiar with a DFS algorithm as an algorithm which traverses a graph by starting at a root node and examining each branch as far as possible before backtracking to examine other branches. This is more computationally efficient than using other graph traversal algorithms, such as a breadth first search algorithm, as only the current path i.e. from the root node to the current node, is stored in the memory.

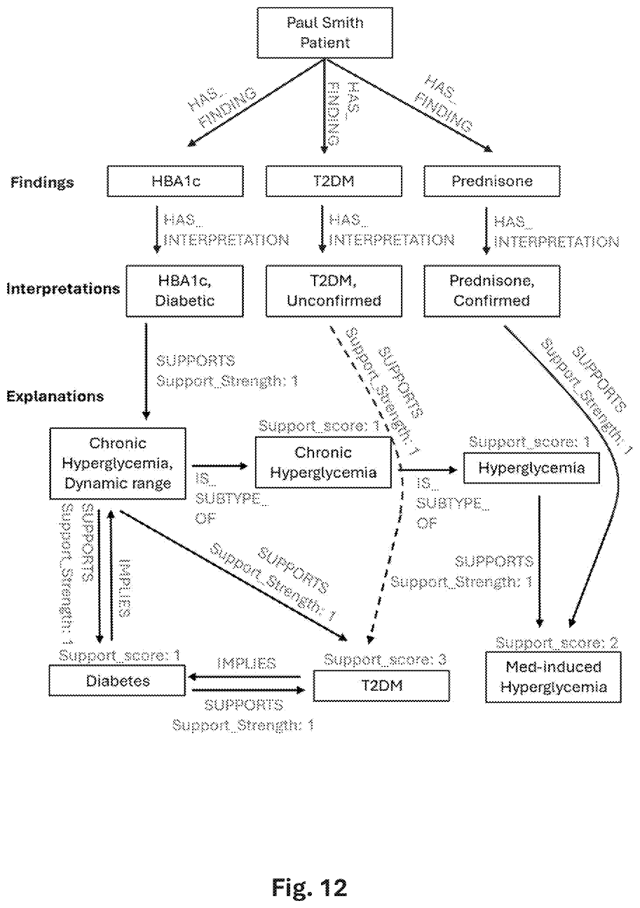

FIG. 12 shows an example patient-specific-KG or CHG generated for a specific patient. The CHG comprises the patient data in a global input node or patient node at the top of the graph. The patient node branches out to three nodes in the first layer of the CHG. In this example, the first layer comprises a finding layer and therefore the edges between the patient node and the nodes comprised in the first layer i.e. finding nodes, are labelled as “has_finding”. It will be understood that the finding layer may comprise fewer or more than three nodes and the number of finding nodes will depend on the number of data points comprised in the patient data that satisfy the rules and criteria embedded in the finding nodes. These rules and criteria may for example include one or more “if-then” statements, which match the data points with the appropriate finding node.

In the example patient-specific-KG of FIG. 12, each finding node branches out to a node comprised in the second layer of the CHG. The second layer comprises an interpretation layer and the nodes house (e.g. store) the interpretations for the values of the findings comprised in the finding layer.

The interpretation nodes branch out to nodes in the third layer of the CHG i.e. the explanation layer. The interpretations are reasoned over by exploiting the global-KGs relationships and the embedded rules to arrive at the best explanations for the patient's clinical state. The second and third layers are connected by edges labelled as “supports” or “rejects” (not shown). For example, in FIG. 12 the Hb1Ac test result is interpreted as a diabetic condition which “supports” an explanation of e.g. chronic hyperglycemia in the diabetic range. The explanation layer comprises a plurality of interconnected nodes that captures the complexity and interdependence of possible explanations for the patient data. These explanations can be extracted as a list of health problems or health risks to be sent to the patient and/or clinician in the form of an electronic message.

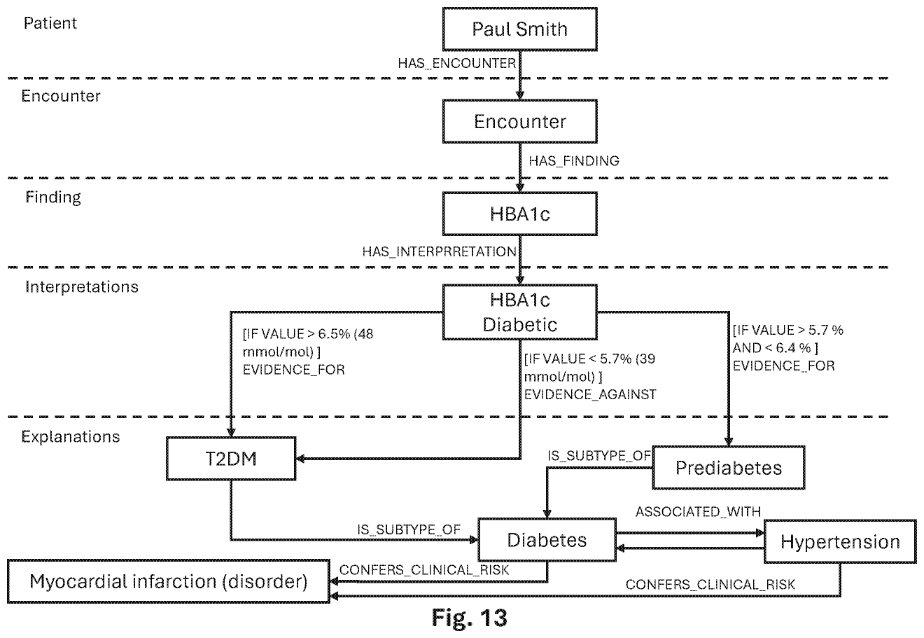

Another embodiment of the CHG shown in FIG. 12, is shown in FIG. 13. In this embodiment, an encounter layer comprising encounter nodes (described above with reference to FIG. 11) is included before the first layer of the CHG and the second and third layers of the CHG, e.g. the interpretation layer and the explanation layer, are connected by edges labelled as “evidence_for” or “evidence_against” (instead of “supports” or “rejects” as shown in FIG. 12). For example, elevated HBA1c levels indicate hyperglycemia and poor blood sugar control over time. High or low levels of a factor or condition can be determined by the use of comparative thresholds. For example, a test result showing an HBA1c value of greater than 6.5% is a diagnostic criterion i.e. “evidence for”, diabetes e.g. Type 2 Diabetes Mellitus (T2DM). Alternatively, low HBA1c levels provide support against the presence of hyperglycemia, suggesting good blood sugar control. A test result showing an HBA1c value below 5.7% is considered normal and provides “evidence against” a diagnosis of diabetes or prediabetes.

The nodes in the explanation layer that are related by causative or correlative risk may be linked e.g. using a Machine Learning Model that looks for semantically correlated concepts in the first information and links related concepts in the explanation layer. An explanation node may be linked to one or more other explanation nodes via edges labelled as “confers_clinical_risk” or “associated_with”. The links between the explanation nodes may be bi-directional (e.g. there may be reverse edges present).

A causative risk involves a direct cause-and-effect relationship between a factor and a health outcome. For example, an explanation node comprising an “explanation” of diabetes may be linked to an explanation node comprising an “explanation” of myocardial infarction via an edge labelled as “confers_clinical_risk” because diabetes is a primary cause of heart attacks.

A correlative risk involves associations between factors that do not imply a direct causal relationship. For example, an explanation node comprising an “explanation” of diabetes may be linked to an explanation node comprising an “explanation” of hypertension via a bi-directional edge labelled as “associated_with” because diabetes and hypertension often occur together due to shared underlying risk factors such as obesity, but one disease does not cause the other. The explanation node comprising an “explanation” of hypertension may also be linked to the explanation node comprising an “explanation” of myocardial infarction via an edge labelled as “confers_clinical_risk” because high blood pressure is also a direct cause of heart attacks. Accordingly, each link to a different part of the data structure may have a specific meaning describing a specific type of data relationship, as illustrated above. Use of a specific different type of link enables more complex data relationships to be escribed more easily to the data structure.

The edges between the nodes result in a more granular understanding of a patient's condition risk as they provide a more detailed insight into how various factors e.g. genetic or environmental, contribute to a patient's risk for a particular health condition. This detailed understanding enables healthcare interventions to be better tailored to a patient, including more personalised treatments for a patient's condition or more accurate preventative measures for a patient at risk of a condition.

The structure of the CHG, for example as shown in FIG. 12, therefore leads to explainable diagnostic querying of the global KG as the reasoning of the underlying AI models is presented within the graph structure of the CHG. This is an advantage of the aforementioned deterministic nature of the solution presented herein.

Additionally, the CHG can be easily updated by updating the patient data and triggering the global KG to be queried again to output a new, updated CHG.

Generating a CHG for each patient using the same global-KG (or CDDT or DDT) allows a population to be stratified into health status levels. For example, the population can be divided into four health status levels including “Healthy” (Level 1), “Acute Care” (Level 2), “Chronic Care” (Level 3) and “Hypercare” (Level 4). A patient's assigned health status, when considered in combination with the patient's health problems or health risks retrieved from the CHG, may change the patient's healthcare access, needs and/or health recommendations. The result of the CHG is therefore enhanced by such a classification of the population.

In an example, to improve the precision of a CHG for patient diagnosis and treatment planning, statistics indicative of the prevalence of a disease or health condition in a population can be combined with the CHG explanations. Prevalence refers to the proportion of a population that is affected by a particular disease or health condition at a specific point in time or over a specified period. Prevalence statistics thus help to understand how widespread a disease or condition is within a population, aiding public health officials in assessing disease burden, planning interventions, and allocating resources effectively.

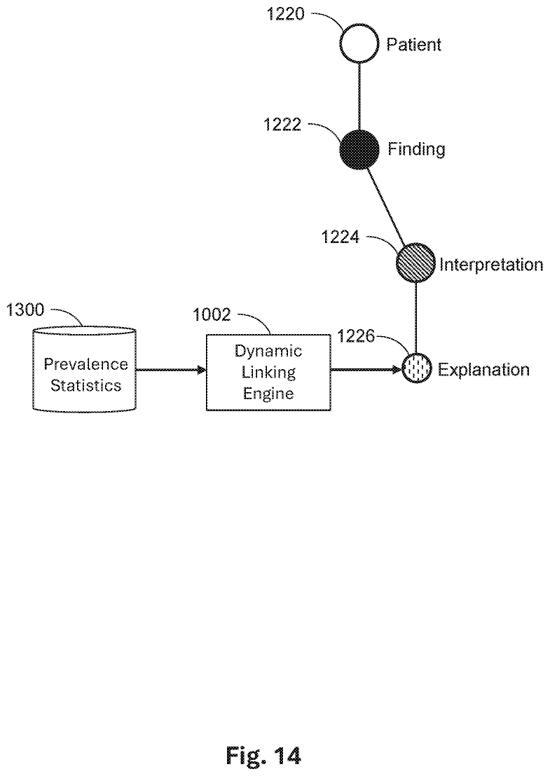

With reference to FIG. 14, prevalence statistics 1300 for a particular disease or health condition can be added as a property to one or more explanation nodes 1226 of a CHG. Prevalence statistics 1300 can be sourced from one or more prevalence statistics knowledge bases, such as the World Health Organization (WHO) or journals such as The Lancet, New England Journal of Medicine (NEJM). For some diseases or health conditions, such as diabetes, prevalence statistics may be sourced from disease-specific knowledge bases such as the American Diabetes Association (ADA), In an example, a single knowledge base containing the information from all available sources, e.g. the WHO, the ADA, journals etc., may be developed (e.g., compiled) by clinicians. This is useful for consolidating data into a uniform format, as different sources may report prevalence statistics differently for different demographics.

The prevalence statistics 1300 that are relevant to a particular explanation node 1226 are imported to the explanation node 1226 using the Dynamic Linking Engine 1002 described above. The Dynamic Linking Engine 1002 leverages one or more prevalence statistics knowledge bases to fetch (e.g. import) the most relevant data (e.g. prevalence statistics) for a given explanation node.

Using prevalence statistics is useful for understanding the scope and impact of various health conditions. By accurately measuring how common a disease is within a specific population, healthcare providers can better identify trends and disparities in health outcomes. For instance, a high prevalence of Type 2 Diabetes Mellitus (T2DM) in younger populations may prompt targeted interventions aimed at lifestyle modifications and early detection programs. Conversely, understanding the prevalence in older populations can assist in managing chronic conditions and improving the quality of life.

Moreover, prevalence data can influence policy decisions, guiding the allocation of funding and resources to areas where they are most needed. This data can also support research initiatives, providing a foundation for studies that aim to uncover the underlying causes and risk factors associated with different health conditions.

In the context of clinical practice, incorporating prevalence rates into computational models, such as the CHG, increases the precision of CHGs used for patient diagnosis and treatment planning. This increased precision allows for a more personalized approach, where the patient's individual risk profile is considered alongside broader epidemiological data, resulting in more accurate and effective healthcare delivery.

Therefore, the implementation of prevalence statistics within health graphs (e.g. CHGs) enriches the data and fosters a more comprehensive understanding of patient health, ultimately leading to better clinical outcomes and optimized healthcare systems.

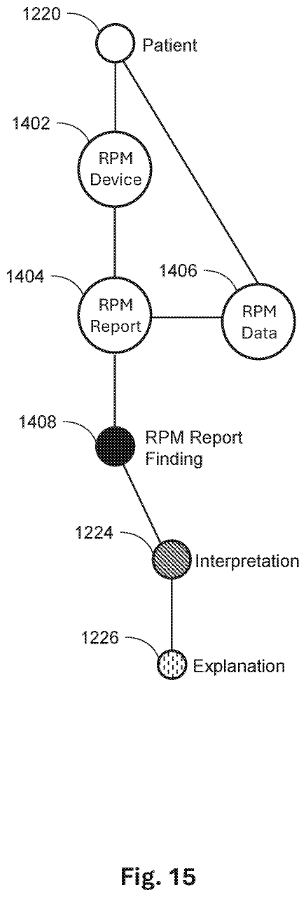

In an example, the accuracy of the CHG generated for a patient can be improved using remote patient monitoring (RPM) (i.e. non-encounter data) via e.g. wearable devices, to collect and transmit patient health data to the root node or patient node of the GKG. As described above, the CHG can be easily updated in this way by triggering the GKG to be queried again to output a new, updated CHG when additional patient data is provided. The collection of real-time health data thus improves the management of acute and chronic conditions than relying on data collected during clinical visits only. An example of using remote patient monitoring data to generate a CHG is shown in FIG. 15.

In the example of FIG. 15, data is collected from a remote patient monitoring (RPM) device, for example a continuous glucose monitoring device. RPM data is flagged with an appropriate code to indicate the data origin. The RPM data is input to the global root node or patient node 1220 and subsequently input to a ‘RPM device’ node 1402 based on the code. The RPM data is then passed to a subsequent node, the ‘RPM Report’ node 1404, wherein embedded rules and criteria are used to summarise the RPM data over a predefined time period. The patient node 1220 may receive additional data from other RPM devices and this additional data may be input to a separate node such as ‘RPM Data’ node 1406. The additional data is then also passed from the RPM Data node 1406 to the RPM Report node 1404 to be summarised according to pre-defined rules and criteria.

The summarised data from the RPM Report node 1404 is then parsed through the Finding layer 1408, the Interpretation layer 1224 and the Explanation layer 1226 according to method 800 described above. In this example, the Finding node comprises an ‘RPM Report Finding’ node 1408.

The use of RPM data in addition to patient data collected during a clinical visit, increases the volume and frequency of collected data points for a particular health condition. This in turn increases the accuracy of the generated CHG and leads to more informative and accurate insights of a patient's health condition and disease risk.

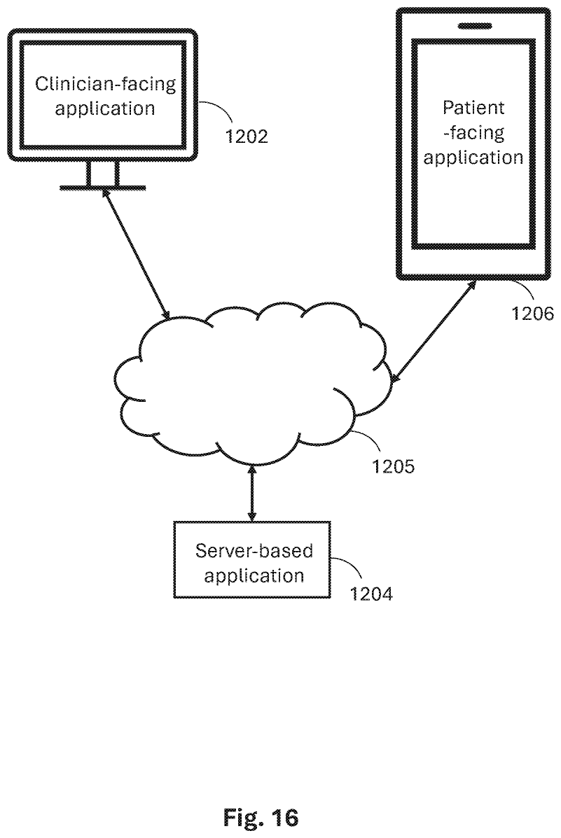

Turning now to other embodiments, the systems described herein provide new opportunities for interconnected digital healthcare systems. FIG. 16 illustrates a digital healthcare system according to some embodiments herein.

In this embodiment, there is a clinician-facing application 1202, a patient-facing application 1206 and a server-based application 1204. All of these applications are connected by a wide area communications network 1205 such as the Internet. In this sense, an “application” is a computer program or process that can be run on a computer node such as the computer node 100, described above. There may be a different computer node 100 associated with each of the applications described herein. Alternatively, they may all run on the same computer node 100. The applications may be encoded in the form of an “app” or any other type of interface.

The clinician-facing application 1202 and/or the patient-facing application 1206 may be configured to run on any type of computer system, such as for example, a desktop computer, a laptop computer, a tablet, a mobile phone, or any other type of computer system. The server-based application may be cloud-based (as illustrated in FIG. 16), or housed across one or more servers at a particular location (e.g. such as a hospital or other medical base). The server-based application may have an interface that can be accessed by human engineers or clinical practitioners, e.g. for maintenance, or updating of the systems thereon.

The server-based application has access to a global-KG as described in the embodiments above.

The patient-facing application is designed to be accessed by a patient. As described in more detail below, the patient-facing application 1206 may have one or more mechanisms for a patient to input personal data, symptoms, changes to their health or circumstances, or any other information that may be relevant to their health outcomes. As described in more detail below, the patient-facing application 1206 may have chat-based functionality (e.g. enabled via a LLM, various examples of which were cited above) to enable patient inputs to be provided verbally. In some embodiments, the patient-facing application 1206 therefore has speech to text functionality to enable the patient to provide the patient data verbally.

The clinician-facing application 1202 is designed to be accessed by a clinician (e.g. healthcare professional, doctor, nurse, surgeon etc). As described in more detail below, the clinician-facing application 1202 may have one or more mechanisms for a clinician to input data about the patient, such as symptoms, changes to their health or circumstances, or any other information that may be relevant to their health outcomes.

In some embodiments, the global-KG on the server-based application 1204 may be accessible by both the clinician-facing application 1202 and the patient-facing application 1206 (for example, via an API). As noted above, the global-KG described herein allows for particularly high levels of inter-operability and automation.

In some embodiments, the patient-facing application 1206 is configured to receive, in a first user input, first patient data, and send the first patient data to the server-based application. The server the server-based application is configured, upon receiving the first patient data, to send the first patient data to be parsed through a global KG, comprising a plurality of sub-KGs, each sub-KG relating to a different health condition (as described above). The server-based application 1204 is then configured to receive a first clinical insight as output from the global-KG, and send the first clinical insight to the clinician-facing application. The clinician-based application is then configured to display a first update, in response to receiving the first clinical insight.

The first patient data may be provided by the patient in the first user input (which may be verbal, or input via a keyboard or similar). In this embodiment, the sending of the first user input triggers the global-KG, accessible by the server-based application, to re-process the data for the patient. In this sense, the full data record for the patient may be re-processed, or just the newly received first patient data.

In response to the re-processing, a (new) clinical insight for the patient may be determined and this determination triggers a message to be sent to the clinician-facing application, for display of the first update on the clinician's device. The first update may comprise the first clinical insight, a message to the clinician (e.g. indicating that a new insight is available for the patient), a recommendation for the patient in view of the first clinical insight, or any other information that may be derived or triggered from the first clinical insight. In this way, data input by the patient may be processed in an automated manner, and a new first clinical insight may be produced and passed to the clinician without any manual intervention on behalf of either the clinician or the patient (beyond the first user input).

The process can be triggered in a reciprocal manner. For example, the clinician-facing application may further be configured to receive in a second user input (e.g. provided by the clinician), second patient data, and send the second patient data to the server-based application. The server-based application may then be configured upon receiving the second patient data, to send the second patient data to be parsed through the global-KG, receive a second clinical insight as output from the global-KG, and send the second clinical insight to the patient-facing application. The patient-based application may then be configured to: display a second update for the patient, in response to receiving the second clinical insight.

Thus, second patient data, input by a clinician (e.g. during or after a consultation, or in response to new test results being received etc) may also trigger the global-KG to re-parse or reprocess the patient record (or just the new data, e.g. just the second patient data). Obtaining a new output of (e.g. a new clinical insight from) the global-KG for a patient, may trigger the insight to be sent to the patient-facing application 12062. In this way, new information input by the patient may i) trigger the global-KG to be re-queried for the patient and ii) may trigger an update in the clinician-facing application 1202. Conversely, new information input by the clinician may i) trigger the global-KG to be re-queried for the patient and ii) may trigger an update in the patient-facing application 1206.

The clinician-facing application 1202 may have further functionality. For example, the clinician-facing application 1202 may enable the clinician to model or simulate changes to the patient data and patient outcomes if different data is submitted, or certain values are changed. In other words, the clinician can run simulations for different scenarios for the patient. The aforementioned deterministic nature of the global-KGs herein are advantageous in this respect as the results of the simulations are repeatable. Furthermore, the explainable nature of the patient-specific KGs described above, mean that the simulations produce meaningful output for the clinician, thus explaining why a particular change leads to a particular result.

These features can also be used to track and/or explain changes in the clinical insights produced for a patient over time. For example, the clinician may input a real or measured change for the patient, and this can be processed in a similar manner, with the changes that this produces to the diagnosis or prognosis for the patient being highlighted and explained to the clinician.

Thus, the clinician-facing application 1202 may be further configured to receive in a third user input, third patient data (either hypothetical data if the clinician is modelling/simulating a scenario, or real data if the patient data has changed over time), and send the third patient data to the server-based application. The clinician-facing application may be further configured to receive a third clinical insight as output from the global-KG, and display a comparison of the third clinical insight, with one or more previous clinical insights (e.g. historical patient data, or other theoretical patient data) obtained for the patient.

The third clinical insight (or any other clinical insight herein) may be displayed as a patient-specific KG (as described above). As noted in earlier embodiments, the patient-specific KG comprises a subset of nodes from the global-KG that were activated when the third patient data was parsed through the global-KG. In some embodiments, the comparison may be displayed or highlighted as a change to the patient-specific KG. For example, some nodes may be greyed-out indicating that they are no longer activated by the change in the patient data, and/or some nodes may be further highlighted if they are newly activated. This is merely an example however, and a comparison between two patient-specific KGs may be implemented in a wide range of different ways.

Thus, by inputting theoretical or test values into the global-KG herein, a patient-specific KG (CGH) can be produced that highlights the risk factors associated with the test value. In this way, the CGH can be used both for identifying health problems of a patient, and for identifying potential health risks using a logical and explainable graph structure.

Thus, embodiments herein provide enhanced interoperability between the patient-facing application 1206 and the clinician-facing application 1202. In some embodiments, both applications have access to the CGH of the patient associated with the patient-facing application, and both receive updates thereto triggered by new or changed patient information input by either the patient or clinician on their respective applications.

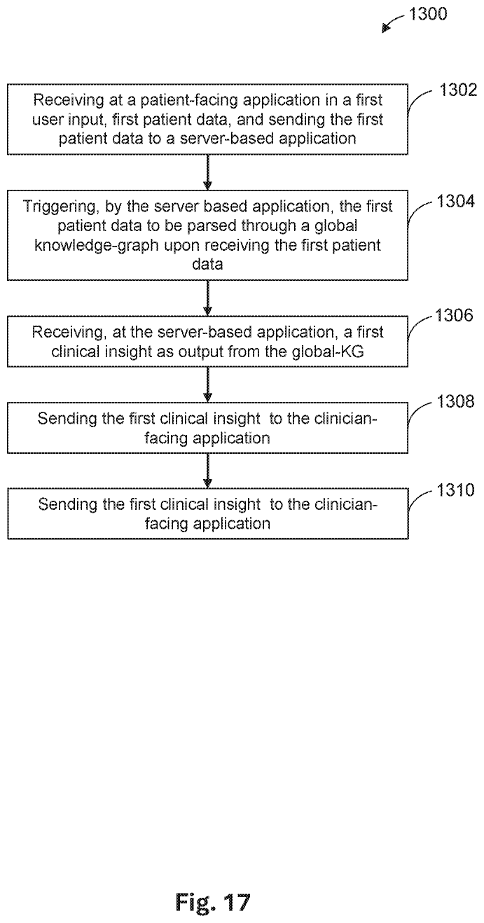

FIG. 17 shows a method 1300 in a digital healthcare system according to some embodiments herein. The method 1300 is computer implemented and can be performed by one or more computing nodes 100 as described above.

In a first step 1302, the method 1300 comprises a patient-facing application 1206 receiving in a first user input, first patient data, and sending the first patient data to a server-based application 1204. In a second step 1304, the method 1300 comprises the server based application1204, upon receiving the first patient data, triggering the first patient data to be parsed through a global knowledge-graph (GKG) comprising a plurality of sub-KGs, each sub-KG relating to a different health condition. In a step 1306, the method 1300 then comprises the server-based application 1204, receiving a first clinical insight as output from the global-KG. In step 1308, the first clinical insight is sent to the clinician-facing application 1202 and in step 1310, the clinician-based application, in response, displays first update, in response to receiving the first clinical insight. These steps were described above with respect to the system diagram in FIG. 16, and the detail therein will be understood to apply equally to the method embodiment.

In some embodiments, the patient cannot directly alter information in their EHR to avoid introducing errors to the underlying data, but the patient can provide additional data (e.g. such as but not limited to sleep pattern data, reactions to a medical treatment, or any other health relate data) that is then input to the global input node of the GKG to output an updated CGH by traversing the additional information through the GKG. This provides the clinician with an up to date and more accurate overview of the patient's medical status based on information that might not otherwise be captured in a standard medical appointment. In another example, additional patient data may comprise measurements of a level of pollution in the patient's daily environment.

An advantage therefore of processing patient data or medical data using AI techniques to produce the CGH as described above, is that the CGH may recognise health risks that would not otherwise have been identified by a clinician. Moreover, the almost instantaneous update of a patient's CGH enables clinicians to use telemedicine more effectively and efficiently to provide support and health care to patients at a distance. The interoperability of the clinician- and patient-facing applications also enables the patient to follow their health journey and be more acutely aware of potential risk factors.

Furthermore, the G KG is able to take all information available into account when making a diagnosis, and is not biased, e.g. towards the patient's latest symptoms.

In some embodiments herein there are provided one or more pre-built and optimised AI models that are used to generate insights for a patient-facing application and a clinician-facing application. These AI models may comprise the AI models used to generate the global KG (or CDDT or DDT), the patient-specific KG (or CHG) and the ML model comprised in the HDLL, and the group of these AI models is referred to herein as the “Health AI Engine”.

The clinician-facing application 1202 can effectively act as a clinical assistant to enable clinicians to provide high quality, prevention-focused healthcare and address the shortage and knowledge gap between PCPs and specialists, by augmenting clinical intelligence and reducing administrative burden of care teams.

The clinician-facing application 1202 provides AI-powered clinical decision support, enabling the state of healthcare to become more proactive, preventative, and personalized to each individual, based on the best global standards of care.

While guiding clinicians to the next-based action grounded in the leading standards of care, the clinician-facing application 1202 also educates the clinician through in-the-moment knowledge nuggets and by surfacing contextualized literature.

The clinician-facing application 1202 will enable three (3) core capabilities for clinicians:
1. AI-powered clinical decision support & administrative unburdening: the clinician-facing application provides clinical insights and suggestions at the point of care to guide patient evaluation, diagnosis, treatment and follow-up. It assists clinicians with administrative tasks to free up clinicians to spend time on the patient interactions that matter.
2. Care team and patient collaboration: Through the clinician-facing application, Primary Care Physicians (PCPs) will be able to collaborate seamlessly with other members of the care team (specialists, nurses, care coordinators, etc.) through synchronous and asynchronous communication, as well as interact with their patients to educate and guide them through their care plans.
3. A novel UX for high-dimensional, high-velocity information: the clinician-facing application 1202 may use a gaming-inspired touch user experience, combined with a more natural conversational interaction to enhance how clinicians consume information in an intuitive, engaging way.

In some embodiments, the clinician-facing application 1202 meets the following requirements:

1. AI-enabled clinical decision support & administrative unburdening
a. AI Insights—AI-enabled clinical decision support that provides personalized care recommendations (sourced from the Health AI Engine). Insights are surfaced in context of the specific patient and the clinician's workflow (e.g., next best action for diabetes treatment will be surfaces while exploring the Diabetes condition). Examples of AI insights include:
   i. Differential and missed diagnoses
   ii. Recommended evaluation plan
   iii. Recommended treatment plan
   iv. Care gap identification and closure recommendations
   v. Drug interaction alerts
b. Incorporation of clinical expertise—the AI insights for each clinical condition are informed by leading clinical guidelines, literature and centers of excellence.
c. Command and Control Center (CCC)—an interactive population view that visualizes at-risk patients (Time-sensitive targets and Clinical-sensitive targets) within the population, and recommends targeted interventions). The CCC may include proactive notifications to the clinician, as well reactive notifications (when queried by the clinician)
   i. Time-sensitive targets (TST)—Display population of otherwise healthy individuals with risk factors (based on labs, genetics, environment, behavior, etc.) that may lead to poor health outcomes.
   ii. Clinical-sensitive targets (CST)—Display individuals that are already in the sick care system that have risk factors that may exacerbate existing conditions or cause new ones.
d. Use Color to derive Insights—Clinicians will be able to use color spectrum visualization to derive patient-level and population-level insights (e.g., patient health progression over time across various levels of care, from less critical to more critical). The use of color (including intensity and vibrance) is intended to allow users without a deep data science background to analyze data and identify patterns
e. Health list viewer—provide a view of each patient's comprehensive problem list, including risk factors. The health risk viewer may:
   i. Be comprehensive: Show each patient's comprehensive set of problems, conditions and risks
   ii. Show interrelationships between each condition (based on leading medical ontologies and literature)
   iii. Be filterable—to show user-specific perspectives of the Health List (e.g., by specialty, by organ system)
   iv. Show alerts—to notify clinicians which conditions may have new information (e.g., abnormal labs or images), or critical insights
   v. Allow for further navigation into the Condition Explorer by selecting a specific condition in the Health List
f. Condition Explorer—Rather than providing disjointed facts to the clinician, the Condition Explorer will allow clinicians to view details of each condition, and use AI to surface the relevant information, oriented around the specific condition being reviewed
   i. Widgets—the condition explorer will have widgets that provide patient-related information, contextualized for the specific condition. These widgets will be intelligently arranged (e.g., criticality of information, relevance to the particular condition, user preference) to give the clinician just-in-time information that is actionable. Widgets may include (but not limited to):
      1. Visits
      2. Labs
      3. Findings
      4. Imaging
      5. Current medications
      6. Differential diagnoses
      7. Care plan recommendations
      8. Patient reported outcomes
      9. Insights from real-world evidence
   ii. Informational & data input—Some widgets in the Condition Explorer will be informational (e.g., information about imaging reports), while others will require input from the user (e.g., change medications)
g. Knowledge Center—Dynamic content, generated based on the current clinician activity, including up-to-date guidance on best practices, new research, or observational breakthroughs. The Knowledge Center will surface insights to the clinician just-in-time, in context of the specific patient and condition being explored.
h. Prepopulation of data—As a digital assistant, the clinician-facing application will help reduce the administrative burden that plagues today's clinicians by intelligently prepopulating information in the patient's record and care plan, that can then be confirmed by the clinician before finalized (e.g., lab orders, med orders, consult notes)
i. Integration with EMR—documentation and orders placed within the clinician-facing application 1202 may be pushed electronically to the patient's electronic medical record (EMR), without requiring the clinician to manually make the updates in the EMR.

2. Care team and patient collaboration
a. The Lounge (care team collaboration)—Real-time connectivity with colleagues and specialists to collaborate on treatment plans, seek medical guidance, and share consults. The Lounge will allow for synchronous and asynchronous messaging, as well as sharing of video clip “perspectives” (e.g., a screen recording of the PCPs findings using the clinician-facing application) to facilitate team consults and discussion
B. Connection with patient-facing application—Clinicians will be able to view information provided by patients via patient-facing application (e.g., patient reported outcomes), push information to patients via patient-facing application (e.g., care plans, patient education), and enable synchronous and asynchronous communication (e.g., messaging, video chat)

3. A novel UX for high-dimensional, high-velocity information
a. Virtual avatar—the clinician-facing application may comprise a conversational avatar, powered by generative AI, to allow touch-free, keyboard-free user interaction. An “avatar” includes a hyper-realistic, customizable human character, who both speaks directly to clinicians and emotes when actively listening
   i. Inputs—Similar to the patient-facing application, the clinician-facing application's AI concierge achieves sensor-based perception through audiovisual sensors
b. Processing—the inputs above may be combined with any other appropriate information (e.g., patient context) for insight generation.
c. Gaming-inspired UX—An engaging user interface that makes it easy for physicians to review patient information and consider treatment options
   i. Heads up display—The interaction overlay for smart features (AI Insights, an intuitive way that provides consistency, such that the user can find information in a consistent location, that is accessible but unobtrusive
   ii. Information landscape—the dynamic area in the center of the app screen where key, contextual information is displayed (e.g., patient's health list, labs, medication, Digital Twin visualization)
   iii. Dynamic navigation—AI-powered layout configuration of the Condition Explorer, with the widgets organized in an intelligent way (e.g., based on relevance, or temporality). More details on the Condition Explorer are included below.
D. Digital Twin Visualization
   i. The Digital Twin is the basis for patient health personalization, prediction, and care plan recommendations. Each interaction (via the patient-facing application or from the healthcare system) enriches the twin, further improving personalizations and recommendations
   ii. To accomplish this a compelling visualization of the patient's Digital Twin (pulling from the Health AI Engine Digital Twin Models) may be provided. The visual may allow clinicians to navigate the Digital Twin and explore by various parameters (e.g., organ system, anatomical location). AI algorithms will enable clinicians to predict patient outcomes based on multifactorial criteria (e.g., care plan compliance, genetic predisposition), provide the rationale for the predictions, and provide next best action to achieve desired patient outcomes
   iii. The Digital Twin may start with a limited perspective, and as more patient information is collected over time, the Digital Twin becomes richer and more predictive

The patient-facing application enables a direct connection to—and health companion for—each patient, working tirelessly to deliver on the promise of proactive, preventative care by nurturing and empowering each individual to live their healthiest life.

The patient-facing application serves as a trusted, empathetic personal health confidant, combining speech and sensory perception with an approachable, human-like avatar to make interactions more natural, more accessible, and more conversational.

The patient-facing application listens, earning permission and trust, to truly understand patients. It is attentive to adoption and uptake, ensuring to provide early benefits and reliable outputs in order to establish credibility and drive further desirability. It uses each interaction to enrich each patient's digital twin, allowing it to demonstrate a deeper understanding of what they want to achieve in their lives, reinforcing its genuine and nurturing nature.

The patient-facing application focuses on the journey and how each patient wants to reach their health goal. It uses personalization to deliver AI-powered guidance and education alongside proactive outreach and support with the compassion of a real person.

The patient-facing application relieves the patient from unnecessary administrative tasks and burden. The patient-facing application integrates any “formal” access to the provider system into a friction-free, natural experience considerate of the patient's and interaction context. It delivers medication reminders, seamlessly handles scheduling in the background, and even explains new medications or lab results, the key insights, and the next steps to take. Furthermore, these interactions reinforce care plans and clinical guidance, serving as a direct linkage to and extension of the care team, creating richer and more genuine relationships between patient and care team.

The patient-facing application is built upon three (3) core tenets:
1. Deliver a virtual human health “confidant” experience The patient-facing application moves beyond keyboard, mouse, and touch and provides more natural interactions through speech, ongoing conversation, and sensor-aided perception
2. AI-powered guide to usher patients on their journey to increased health span The patient-facing application focuses on the patient's journey, which is the way they want to reach their own health goals. It builds trust with the patient, then uses conversation, tactful probing, and behavioural techniques to tailor the journey based on the patient's sensitivities, motivations, and the barriers in their way. The patient-facing application refines its guidance and content with every interaction, increasing its stickiness and likelihood to motivate change.
3. Create an engaging relationship between patient and care team The patient-facing application connects PCPs with their patients, reinforcing care plans and clinical guidance, while providing insight and access to the latest data and patient progress to the entire care team (spanning health coaches, population health managers, care coordinators, and specialists, among others).

The patient-facing application has several key experience considerations:
1. Gamified Patient Journey
   a. Develop a patient-centric application that transforms “traditional” healthcare activities into engaging, game-like experiences: Implement gamification elements to motivate users to adhere to treatment plans, medication schedules, and healthy lifestyle choices.
2. Personalization and Progress Tracking
   a. Personalization: Incorporate features for personalized gaming experiences based on individual health goals and preferences.
   b. Tracking: Include mechanisms for users to track their progress and achievements within the application.
3. Segmented user group (e.g., age-specific) interface
   a. Design an intuitive interface to ensure accessibility for and comprehension by patients of various backgrounds (e.g., ages, technological proficiency, health literacy).
   b. All versions aim to deliver a seamless and enjoyable user experience, catering to a diverse user base.
4. Integration with health data and connected devices (e.g., wearables)
   a. Enable integrations for various health data sources, allowing users to track and monitor their health progress.
5. Digital Twin Visualization
   a. Internally, the Digital Twin is the basis for individual health personalization, prediction, and guidance and each interaction enriches the twin, further improving personalisation and recommendations. Externally, the Digital Twin is an to provide data for improved direction.
   b. Create a compelling visualization of the patient's Digital Twin (pulling from the Health AI Engine Digital Twin Models), that allows them to view predicted outcomes based on multifactorial inputs (e.g., positive effect of more sleep, negative impact of non-adherence to the care plan). The visual can convey the expected outcome, the rationale for that outcome (education), and the steps to follow to achieve the desired outcome.
   c. The Digital Twin may start with a limited perspective, and as the patient-facing application collects more patient information over time, the Digital Twin becomes richer and more predictive. The visual helps compel this data entry, showing the “fidelity” that comes from the addition of information.
6. Educational Content
   a. Include educational content—in conjunction with personalization, gamification, and progress tracking—to inform users about their conditions, treatment options, and healthy living practices.
7. Compatibility
   a. Ensure cross-platform compatibility, supporting both iOS and Android devices.
   b. Optimize the application for various screen sizes and resolutions.
8. Privacy and security measures
   a. Implement robust security measures to safeguard patient data and patient privacy to maintain confidentiality.
   b. For devices (as an IoT Hub): Ensure compliance with relevant healthcare data protection regulations and standards.
9. Scalability and maintenance
   a. Develop a scalable solution that can accommodate future updates and additional features.
   b. Provide ongoing maintenance and support services post-launch.

Ultimately, the patient-facing application's individual health journey creation combined with proactive prevention will simplify patients' lives and help them to become and stay healthy.

The design of the patient-facing application meets the following requirements:

### 1. Deliver a Virtual Human Health “Confidant” Experience

The patient-facing application is a “Virtual Robot,” a human-like, on-screen avatar that listens and observes the Patient. It responds with helpful insights and guidance generated from the global-KG delivered using conversational, natural speech and with the appropriate gestures. While the patient-facing application feels like talking to a trusted companion, it is fully powered by AI.

### 1.1 The Patient-Facing Application Inputs

The patient-facing application may achieve sensor-based “perception” through an array of microphones, cameras, connected devices, and environmental measures. The inputs to the patient-facing application may therefore include one or more of the following:
Audio: Captures voice and external sounds, synthesizing both speech and environmental factors (e.g., other voices, background noise), using them to understand a request and build further context around the current situation.
Visuals: Uses cameras to determine patient identity, attentiveness, facial expressions (to inform sentiment), movement (e.g., changing rooms, change in gait/notable limp) and detect environmental factors (e.g., in public? Alone? On the move?)
Connected devices: Uses connected medical devices (e.g., glucometer) and wearables (e.g., smart watch) to capture real-time health information for submission
Additional context: References other, appropriate sensors to complete the user “context.” Examples include: weather conditions, time of day, location, device in use (e.g., phone, smart TV) and even calendar (e.g., patient is late to a meeting)

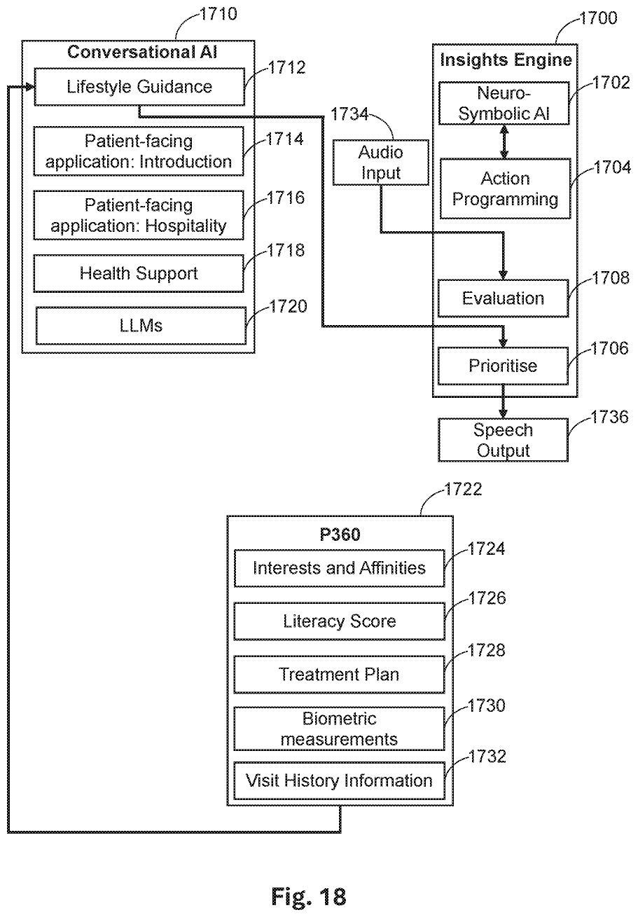

In an example, the patient-facing application 1206 comprises a web application or a mobile application that is powered by a backend module, a Conversational AI agent 1710, that is in communication with one or more other modules for receiving and analysing patient data via user dialogue and for outputting the insights and guidance via the patient-facing application. These other modules comprise an Insights Engine 1700 and the P360 1722 as shown in the block diagram of FIG. 18.

The Conversational AI agent 1710 comprises a Lifestyle Guidance unit 1712, an Introduction unit 1714, a Hospitality unit 1716, a Health Support unit 1718 and one or more LLMs. The Introduction unit 1714, the Hospitality unit 1716 and the Health Support unit 1718 include information for users to learn how to use and/or navigate the patient-facing application 1206, as well as other information to be displayed on a user interface of the web or mobile application. The Health Support unit 1716 may also provide an AI agent (machine learning agent) for responding to user questions, that is powered by the one or more LLMs 1720. The guidance and education described above, is provided to the user using the Lifestyle Guidance unit 1712 in communication of the one or more LLMs 1720 therein. Specifically, the Lifestyle Guidance unit 1712 communicates the guidance and education to the Insights Engine 1700 as further described below.

As described above, the P360 1722 comprises a comprehensive database (e.g. a stored data schema) of all patient-specific health data that is available for the patient in question. The database of the P360 1722 provides a centralised data store from where data can be directly obtained, which enables efficient data retrieval. The P360 1722 comprises the backend infrastructure, databases, and APIs necessary for seamless data handling within the patient-facing application. The P360 1722 may comprise several different patient-specific groups of data such as a patient's interests and affinities 1724, the patient's literacy score 1726, the patient's treatment plan 1728, the patient's biometric measurements 1730, and the patient's clinical visit history information 1732.

The Insights Engine 1700 is responsible for extracting meaningful insights from patient data. The Insights Engine 1700 comprises neuro-symbolic AI models 1702, an Action Programming unit 1704, an Evaluation unit 1708 and a Prioritise unit 1706. The Insights Engine 1700 carries out the functionality of the Dynamic Linking Engine 1002 described above with reference to FIG. 10. The Action Programming unit 1704 links the GKG 706 with patient data and leverages the neuro-symbolic AI models 1702 to generate a CHG for the patient. The neuro-symbolic AI models 1702 may comprise the neural AI model 1014 and the Inference Engine 1016 described above.

As further described below, the Evaluation unit 1708 receives Audio Input 1734 from the patient and analyses the Audio Input 1834, for example by converting the audio data to text using an ETL process 1012. The Prioritise unit 1706 includes an AI router (not shown) that prioritises discussion topics and directs conversational input to the appropriate specialized agents and data sources. By prioritising conversational topics, the outputs of the patient-facing application 1206, such as speech outputs 1736, are also prioritised. Additionally, the Prioritise unit 1706 may receive inputs from the Lifestyle Guidance unit 1712 and prioritise the outputs (e.g. guidance and education) and communicate the outputs to the user via a speech output 1736.

### 1.2. The Patient-Facing Application “Logic” Layer

The patient-facing application “Logic” layer (inputs): Logic layer used to capture and prepare inputs for submission to the global-KG for insight generation. These may include: audio analysis (voice to text), video analysis (e.g., sentiment, gait), device and other sensors (e.g., location, whether involved in an activity/workout, weather conditions).

The patient-facing application “Logic” layer (outputs): Outputs may be received and processed to provide the appropriate output experience (e.g., ensure the patient-facing application's animations, tone, and delivery match the information/education/guidance being delivered).

The patient-facing application “logic” layer may comprise the Insights Engine 1700.

The aforementioned inputs and outputs may be combined with any other appropriate information (e.g., patient's digital twin) for submission to the global-KG by the Action Programming unit 1704 for insight generation. This assumes the creation of appropriate APIs to support the bi-directional flow of information.

### 1.3 The Patient-Facing Application Outputs

Outputs of the patient-facing application 1206:
i. Text-to-voice module: Mechanism for translating outputs from the “Health AI Engine” into spoken responses
ii. Avatar voice: Voice output options-spanning cadence, pitch, and accent-starting in English.
iii. Avatar appearance and animation: Animated, human-like avatar with the natural movement, lips in-sync with voice, and actions informed by the response (i.e., not “beaming” if providing insight about a new diagnosis).
iv. Multi-modal device support: Architected to allow for interactions with the patient-facing application across phones, tablets, computers, TVs, other home devices (e.g., “Google Home”), and AR/VR headsets.

### 2. AI-Powered Guide

The patient-facing application serves as an AI-powered “Sherpa” by capturing patient-driven (or clinician-initiated goals). The patient-facing application creates individual, personalized health pursuits, complete with ongoing guidance, gamification, and encouragement. The patient-facing application enables simple access to essential health system components (such as scheduling appointments and refilling medications) to complement a patient's individual health pursuit.

The patient-facing application 1206 actively monitors progress and intervenes to help remove barriers and support ongoing progress and goal achievement.

2.1 Goal setting: The ability for patient's to provide their goal for the patient-facing application (e.g., “I want to lose weight”). Note: Goal setting may be restricted to health and wellness-related pursuits.

2.2 Personalization: Create each patient's unique pursuit, composed of personalized-content and activities, which together help a patient reach their desired health goal.

2.3. Guidance and gamification: Ongoing guidance and gamification (e.g., challenges, incentives) from the patient-facing application to engage, encourage, and empower individuals.

2.4. Access to appropriate visual aids/tools: Creation of or connection to appropriate tools to assist with care plans (e.g., calorie counter, 3D body model).

2.5. Access to appropriate content: Creation of or connection to engaging, understandable information regarding activity, diet, sleep, Rx adherence, mental health, and behavioral health delivered within each “Pursuit”.

2.6. On-going monitoring: Develop logic to assess progress via active (Patient-reported progress) and passive (mobile health wearable/sensor readings) methods, which then intervenes to adjust the guidance or content to keep patients moving forward.

2.7 Healthcare system access—Enables easy (and intuitive) access to the following:
Patient health profile: Demographics and other baseline health information
Appointments: Schedule, view, update, and cancel
Lab results: View lab results
Imaging: View imaging reports and high-res images from within the patient-facing application
Medications:
   i. Medication information: View medication explanations and instructions
   ii. Medication refills: Request medication refills
   iii. Medication reminders: Send patient reminders when medications are due for a refill.
Telehealth: Enable one-click video consultation
Symptom checker: Enable preliminary self-assessment based on symptoms. Connect to most appropriate resources.
Patient forums: Create forums and affinity groups for patients, based on activities or conditions
Care team messaging: Ability to contact care team, including physician (if appropriate)
Insurance: Add or change insurance coverage
Billing:
   i. Payment processing: Allow patients to pay bills directly through patient-facing application
   ii. Viewing invoices: Allow patients to access bills directly through patient-facing application

3. Creating an engaging relationship with the care team—Bolster proactive prevention via:
a. Individualized care plans: Access to individualized care plans created within the clinician-facing application. These are inclusive of clinical objectives, education topics, and any summary of actions to take for the patient. Note: the clinician-facing application scope may include ability to assess patient's personal goals and ability to meld them with clinical objectives/imperatives.
b. Targeted outreach: Ability to send out health data questionnaires or targeted questions related to care plan progress.
c. Adherence tracking: the patient-facing application may be able to pass through appropriate adherence information to the clinician-facing application regarding both health pursuit and care plan progress, providing a more robust understanding of the patient's progress.

In an embodiment of the present disclosure, there is provided a patient-facing application that is in communication with a platform comprising a plurality of AI models and algorithms which are used to build the global KG and CHG as described above, as well as the ML model that is comprised in the HDLL. The patient-facing application advantageously enables patients to add data that is used to update the computational health graph.

In an embodiment of the present disclosure there is provided a clinician-facing application that is in communication with the server-based application (and thus global-KG) and is also in communication with the patient-facing application. The clinician-facing application comprises an AI-enabled clinical decision support platform that provides personalised care recommendations for each patient to the clinician. These care recommendations may include: differential and missed diagnoses; a recommended evaluation plan; a recommended treatment plan; identification of a care gap and recommendations for closing the gap; and drug interaction alerts.

In order to provide these care recommendations, the clinician-facing application receives guidelines from the server-based application wherein the guidelines are personalised according to the context of the patient. The context may include the location of the patient and therefore the guidelines comprise the most recent and region-specific clinical guidelines available.

Additionally, the clinician-facing application receives the patient-specific KG of each patient, enabling the clinician to view a comprehensive graph of each patient's health conditions and risk factors, including the supporting evidence for each condition.

The clinician-facing application may also receive personalised recommendations on the next best action for evaluation, diagnosis, treatment and follow-up as well as insights on how other similar patients have been treated, their response, and potential risk factors to consider. These features advantageously enable clinicians to consider all possible patient care options.

The clinician-facing application can also send data to the server-based application to indicate which clinical recommendations were accepted, rejected or how they were modified. Subsequently, the server-based application can use aggregated practice patterns to evolve and regionalise the clinical recommendations over time.

The direct communication between the patient-facing application and the clinician-facing application advantageously enables the computational health graph of the patient to be constantly updated by both the patient and the clinician. As a result, the recommendations provided on the clinician-facing application can be continuously updated.

Turning now to other embodiments, it will be appreciated that the methods herein may be embodied in a computer program. For example, a computer program product may comprise a computer readable medium, the computer readable medium having computer readable code embodied thereon. The computer readable code can be configured such that, on execution by a suitable computer or processor, the computer or processor is caused to perform the method or methods described herein (such as the methods 200, 800, 900 and 1300).

A computer program may take different forms, for example, source code, compiled code, executable code, or any other type of code. It will be appreciated that the source code of computer programs may be written in a wide variety of different programming languages, and may take different architectural designs. For example, the functionality described herein may be split across various different sub-routines. Furthermore, the skilled person will appreciate that many different ways of splitting the functionality between the different sub-routines will be possible. The sub-routines may be stored together in one executable file to form a self-contained program. Furthermore, computer programs may call external and/or standard libraries of computer code for performing certain sub-tasks associated with the functionality described herein.

In another embodiment, there is a computer program product comprising non-transitory computer readable media, having stored thereon a computer program as described above. Examples of computer readable media include, but are not limited to: ROM, such as a CD ROM, a semi-conductor ROM or a magnetic recording medium such as a hard disk.

In another embodiment, there is a carrier containing a computer program. Examples of carriers include but are not limited to an electronic signal, optical signal, radio signal, computer storage medium, or similar. The carrier of a computer program may be any entity or device (e.g. hardware) capable of carrying the program. As an example, a carrier may be a computer readable media as described above. In other examples a carrier may be a transmissible carrier such as an electronic or optical signal, which may be conveyed via electrical or optical cable or by radio or other means.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these claims cannot be used to advantage. Any reference signs in the claims should not be construed as limiting the scope.

### Appendix A

### Hemoglobin A1C Interpretation

### Introduction:

The Hemoglobin A1c (HbA1c) lab test is a critical tool in diagnosing and monitoring diabetes and related conditions. This article provides a comprehensive guide to interpreting the results of the HbA1c lab test, linking interpretations to potential causes, and understanding the implications of these results in a clinical context.

### Indications:

The test is used to diagnose and monitor diabetes treatment. It measures the among of the HbA1c in the blood and provides an accurate long-term index of the patient's average blood glucose level.

### Understanding HbA1c Lab Test:

The HbA1c lab test plays a crucial role in diagnosing and monitoring diabetes. The test results are interpreted in ranges, each corresponding to a specific condition.

### Interpreting HbA1c Lab Test Results:

A result of less than 4.0% is interpreted as sustained hypoglycemia.
A result between 4.0% and 5.7% is considered normoglycemia
A result between 5.7% and 6.5% is indicative of sustained hyperglycemia in the prediabetic range
A result greater than 6.5% is interpreted as sustained hyperglycaemia in the diabetic range.

### Causes of Sustained Hypoglycemia:

HbA1c<4.0%

1. A computer implemented method for converting medical data into a graph structure for improved diagnostic querying, the method comprising:
converting first information on a first health condition into a first sub-knowledge-graph, sub-KG, the first sub-KG comprising a KG structure, the KG structure comprising:
a first layer comprising a first root node that is activated based on a first health indicator related to the first health condition;
a second layer of nodes wherein each node in the second layer of nodes indicates a criteria to be applied to the first health indicator indicated in the root node; and
a third layer of nodes, each node in the third layer of nodes being linked to a respective node in the second layer of nodes and corresponding to an interpretation associated with the first health indicator satisfying the criteria of the respective second layer node to which it is linked; and
integrating the first sub-KG into a global knowledge-graph GKG, the GKG comprising a global root node and a first layer of nodes linked to the global root node; and
wherein integrating the first sub-KG into the GKG comprises adding the root node of the first sub-KG to the first layer of the GKG;
wherein converting the first information on the first health condition into the first sub-KG comprises:
prompting a large language model, LLM, to convert the first information into the first sub-KG, wherein a prompt used for conversion provides the first information and a description of the KG structure that the first sub-KG should take, to the LLM.

2. The method of claim 1, further comprising:
converting second information on a second health condition into a second sub-KG, the second sub-KG comprising the KG structure of the first sub-KG; and
integrating the second sub-KG into the GKG, wherein the step of integrating the second sub-KG into the GKG comprises adding the root node of the second sub-KG to the first layer of the GKG.

3. The method of claim 1, further comprising:
comparing the output of the LLM to a gold-standard sub-KG; and
selecting a prompt and/or type of LLM, based on a result of the comparing.

4. The method of claim 1, wherein directional stimulus prompting, DSP, is used to form the prompt.

5. The method of claim 4, wherein retrieval augmented generation, RAG, is further used in combination with the DSP to form the prompt.

6. The method of claim 1, wherein the first layer, the second layer and the third layer of the first sub-KG and the GKG are unidirectional, with no reverse edges.

7. The method of claim 1, wherein the first sub-KG further comprises one or more further layers, the nodes in the one or more further layers representing different explanations for each interpretation of a respective third layer node.

8. The method of claim 1, wherein:
the first health indicator is a laboratory test that can be performed on a patient;
the state of the first health indicator indicates a first range of values of the first health indicator; and
the interpretation of the state of the first health indicator is a medical condition that can be inferred from a laboratory test result with the first range of values of the first health indicator.

9. The method of claim 1, wherein the first health indicator relates to:
a genetic test that can be performed on a patient;
a socio-economic health indicator; or
an environmental health indicator.

10. The method of claim 1, wherein the first health indicator relates to:
a datum collected during a clinical visit; or
a datum collected using a remote patient monitoring device.

11. The method of claim 1, further comprising:
receiving an update to the first information;
converting the updated first information into an updated first sub-KG, the updated first sub-KG comprising the KG structure of the first sub-KG; and
replacing the updated first sub-KG into the GKG in place of the first sub-KG.

12. The method of claim 1, further comprising:
outputting the GKG; and
storing the GKG in a database.

13. An apparatus for converting medical data into a graph structure for improved diagnostic querying, the apparatus comprising:
a memory comprising instruction data representing a set of instructions; and
a processor configured to communicate with the memory and to execute the set of instructions, wherein the set of instructions, when executed by the processor, cause the processor to:
convert first information on a first health condition into a first sub-knowledge-graph, sub-KG, the first sub-KG comprising a KG structure, the KG structure comprising:
a first layer comprising a first root node that is activated based on a first health indicator related to the first health condition;
a second layer of nodes wherein each node in the second layer of nodes indicates a criteria to be applied to the first health indicator indicated in the root node; and
a third layer of nodes, each node in the third layer of nodes being linked to a respective node in the second layer of nodes and corresponding to an interpretation associated with the first health indicator satisfying the criteria of the respective second layer node to which it is linked; and
integrate the first sub-KG into a global knowledge-graph GKG, the GKG comprising a global root node and a first layer of nodes linked to the global root node;
wherein the processor is caused to integrate the first sub-KG into the GKG by adding the root node of the first sub-KG to the first layer of the GKG; and
wherein a first sub-KG of the plurality of sub-KGs is created by conversion of first information on the first health condition into the first sub-KG, the conversion comprising:
prompting a large language model, LLM, to convert the first information into the first sub-KG, wherein a prompt used for conversion provides the first information and a description of the KG structure that the first sub-KG should take, to the LLM.

14. The apparatus of claim 13, wherein the processor is further caused to:
convert second information on a second health condition into a second sub-KG, the second sub-KG comprising the KG structure of the first sub-KG; and
integrate the second sub-KG into the GKG, wherein the step of integrating the second sub-KG into the GKG comprises adding the root node of the second sub-KG to the first layer of the GKG.

15. A data structure for storing medical data for improved diagnostic querying, the data structure comprising:
a global-knowledge-graph, global-KG, comprising, a plurality of sub-knowledge graphs, sub-KGs, each sub-KG comprising a KG structure, the KG structure comprising:
a first layer comprising a first root node that is activated based on a first health indicator related to a first health condition;
a second layer of nodes wherein each node in the second layer of nodes indicates a criteria to be applied to the first health indicator indicated in the root node; and
a third layer of nodes, each node in the third layer of nodes being linked to a respective node in the second layer of nodes and corresponding to an interpretation associated with the first health indicator satisfying the criteria of the respective second layer node to which it is linked;
wherein the global-KG has a global root node and a first layer of nodes linked to the global root node, and wherein the plurality of sub-KGs are integrated into the global-KG by their respective root nodes having been added to the first layer of the global-KG; and
wherein a first sub-KG of the plurality of sub-KGs is created by conversion of first information on the first health condition into the first sub-KG, the conversion comprising:
prompting a large language model, LLM, to convert the first information into the first sub-KG, wherein a prompt used for conversion provides the first information and a description of the KG structure that the first sub-KG should take, to the LLM.

16. A computer program comprising instructions which, when executed by a computer cause the computer to perform the method of claim 1.

17. A computer readable storage medium comprising instructions which when executed by a computer cause the computer to carry out the method of claim 1.
